(19) United States
(12) Patent Application Publication
(10) Pub. No.: US 2009/0157016 A1
(43) Pub. Date: Jun. 18, 2009

(54) Suctioning system, method and kit

(76) Inventor: Carmeli Adahan, Jerusalem (IL)

Correspondence Address: THE NATH LAW GROUP, 112 South West Street, Alexandria, VA, 22314, US

(21) Appl. No.: 11/989,285

(22) PCT Filed: Jul. 24, 2006
(86) PCT No.: PCT/IL2006/000855

§ 371 Date: Jan. 24, 2008

(30) Foreign Application Priority Data
Jul. 24, 2005 (IL) PCT/IL2005/000784
Feb. 1, 2006 (US) 11/344007

(51) Int. Cl. A61M 1/00

(52) U.S. Cl. 604/290; 604/315

(57) ABSTRACT

A suction head in fluid communication with a pump head provides a sub-ambient working pressure to a target area, enabling drainage thereof to a waste container. A passive pressure regulation system enables the working pressure to be maintained at a desired level. A monitoring system for a suction system is also provided.

### FIELD OF THE INVENTION

This invention relates to suctioning systems and methods, and particularly to such systems and methods that apply negative pressure to physiological areas and the like.

### BACKGROUND OF THE INVENTION

There are many medical situations in which applying suctioning to an area of the body may be beneficial, for example: applying a negative pressure to a wound or burn and/or draining the same, draining the trachea, draining fluids from organs and other parts of the body being operated on or being treated, including treatments of a dental nature. For example, negative pressure applied to a wound enhances drainage of fluids or exudate from the wound and promotes tissue growth and wound healing. This method of healing (known as “cupping”) was exercised since the times of ancient Greek physicians until the 19th century.

A number of systems and methods have been developed for providing medical suctioning.

In WO96/05873 an apparatus is disclosed having a porous foamed pad connected by a tube to a canister. A vacuum pump is located within a housing having a recess for receiving the canister. A bacterial filter positioned over the outlet of the canister, and a vacuum pump sucks wound drainage fluids into the canister.

In WO 97/18007 a portable wound treatment apparatus is disclosed, including a housing containing a suction pump and a canister for containing fluids drawn from the wound. The housing is supported on a harness or belt worn by the patient and is connected to a porous dressing at the wound site by a catheter.

In WO 03/016719, a vacuum pump is disclosed having a drive and a disengageable pumping system connected thereto, and a two- or three-chambered canister within which solids, liquids and gases may be separated from one another.

In U.S. Pat. No. 6,648,862 the vacuum desiccator low pressure vacuum pump and trap and is transportable upon a user's person. The device includes a desiccator cartridge containing a fluid trapping agent, and the desiccator cartridge is connected to a vacuum pump member for providing a low vacuum pressure to the interior chamber of the desiccator cartridge. A single passage, one-way, gas/liquid flow pathway connects the inlet port of the desiccator cartridge to an occlusive dressing covering the wound to be drained. A control circuit includes one or more ancillary circuits for controlling operation of the device, such as: a power circuit, a moisture sensor, a timer circuit, a vacuum pressure sensor; and a pressure differential sensor.

In U.S. Pat. No. 5,645,081, a method and apparatus are disclosed, in which a negative pressure is applied to a wound sufficient in time and magnitude intended to promote tissue migration and facilitate closure of the wound.

In GB 2,307,180 (EP 0865304), a porous dressing is applied on a wound from which fluid is drawn into a canister via a catheter using portable suction pump. The pump is contained in housing and is worn on a harness or belt. Overfilling of canister is prevented by a filter contained in the canister and a pressure sensor which detects pressure reductions in tube between canister and pump which occur when drainage liquid covers the filter. A filter is placed between pump and canister, and pressure at the wound site is monitored by a conduit connected to the porous dressing.

In U.S. Pat. No. 4,739,791 a fluid collection container is disclosed, having an inlet connectable to a fluid source and an outlet connectable to a suction source. The container contains a closure member that closes the outlet port when the container is full. The closure member is integrated with a vent valve that is mounted to the container near the outlet.

In WO 03/030966, a system is disclosed for treating a patient with a wound, and comprises a bandage, a receptacle, and a vacuum source. The bandage comprises a cover to seal about the wound and to define a space above the wound in which a vacuum is to be formed. The bandage further comprises a port providing communication with the space. The receptacle is connected to the port to receive exudate from the wound and is provided to be placed below the wound. The vacuum source is spaced apart from the receptacle, is connected to the receptacle.

In WO 03/057070, a ventilated bandage system is disclosed for use with a wound. The system includes a bandage positioned adjacent to the wound to create a sealed environment around the wound. A vacuum source of the system is in communication with the bandage to create negative pressure between the bandage and the wound. The system may also include a first passageway or vent in communication with the bandage and with the surrounding atmosphere, and a second passageway in communication with the bandage and with the vacuum source.

In US 2005/192548, a wound drainage system is disclosed for draining fluid from a wound of a patient. The system includes a drain catheter, and a suction means applies suction at the drain catheter such that fluid is drawn from the wound. While drawing fluid from the wound, a controller periodically increases and decreases in an active manner the application of suction at the drain catheter.

### SUMMARY OF THE INVENTION

Herein, the term “target volume” relates to any body, system or environment to which it is desired to apply a sub-ambient pressure to and/or to drain fluids therefrom. By way of non-limiting example, such a target volume may comprise a body, system, environment, and so on, which may be contaminated with chemical and/or biological and/or other contaminants, regarding which it is beneficial to remove contaminated fluids therefrom.

Herein, the term “medical target volume” relates to any part (external or internal) of the body of a human or animal, regarding which it is desired to apply a sub-ambient pressure to and/or to drain fluids therefrom. By way of non-limiting example, such a target volume may comprise a wound/burn, the trachea, the stomach, intestines, any body cavity (including for example the intra-oral cavity, sinuses, etc.), an organ or other part of the body being operated on or regarding which there is bleeding or regarding which it is beneficial to remove fluids therefrom.

The term “fluid” as used herein includes liquids and/or gases, and may optionally include solids mixed in with said liquids and/or gases.

As will become clearer herein, by “passive” in relation to the pressure regulation system is meant that the pressure regulation system is configured to create and/or control a predetermined sub-ambient desirable pressure, at the target volume, in response to pump flow, i.e., via open loop control, without measuring the vacuum level at the target volume and changing pump flow to compensate for deviation from the desired vacuum level (i.e., in the absence of closed loop control), Thus, the passive pressure regulation system of the present invention is substantially unpowered and/or uncontrolled by an external control unit.

Herein the terms “upstream” and “downstream” are in relation to the general flow direction from the target area (or into the target area) towards the pump head, and beyond. Similarly, the terms “distal” and “proximal” are in relation to the general flow direction from the target area (or into the target area) to the pump head, and beyond.

According to one aspect of the invention, the present invention relates to a vacuum system (or apparatus) for providing a sub-ambient pressure to a target volume, and thus enables fluids to be drained from such a target volume. Accordingly, the invention also includes a corresponding system for draining fluids from such a target volume. In particular, the vacuum system comprises a pump, remote from the target volume and which generates a vacuum thereat, wherein the vacuum level at the target volume may be controlled via any one of a variety of open loop control methods, in which it is not necessary to directly measure the vacuum level at the target volume.

According to this aspect of the invention, a vacuum system for providing a sub-ambient pressure to a target volume, comprises:
a suction head having an inlet arrangement adapted for being in fluid communication with said target volume;
a vacuum pump in fluid communication with said suction head, said vacuum pump being adapted for providing a predetermined working pressure in said system below external ambient pressure of an external environment with respect to said suction head;
a waste container defining a collection volume for collection of materials that may be drained from said target volume in fluid communication with at least one of said vacuum pump and said inlet arrangement; and
a passive pressure regulation system for maintaining said working pressure, said passive pressure regulation system being in fluid communication with said suction head upstream of the said waste container, and said passive pressure regulation system being in one of selective and permanent fluid communication with said external environment.

In some embodiments, the pressure regulation system comprises a venting valve arrangement adapted for enabling ingress of air at ambient pressure into the system responsive to a reduction in said working pressure below a predetermined datum pressure with respect to said air at ambient pressure and for discontinuing said ingress when said datum pressure is restored. The venting valve arrangement may optionally comprise an inlet port having a valve seat and in fluid communication with said ambient air, an outlet port in fluid communication with said vacuum system, and a valve seal biased for sealingly closing with respect to said valve seat by means of a resilient element generating a biasing force of magnitude substantially less than and in a direction generally opposed to a pressure-induced force acting on said valve seal when said working pressure is less than said datum pressure. Further optionally, the venting valve arrangement may comprise an adjustment mechanism for adjusting said datum pressure.

In other embodiments, the pressure regulation system is connected to said suction head permitting selective fluid communication between said target volume and said ambient air, enabling ingress of ambient air into said target volume responsive to a reduction in said working pressure below a predetermined datum pressure and discontinuing said ingress when said datum pressure is restored. Optionally, the pressure regulation system may be in selective fluid communication with an outlet port of said vacuum pump and said external ambient air such as to allow ingress of at least one of fluid from said vacuum pump outlet port and said external ambient air when said working pressure is below said datum pressure.

According to some embodiments of the invention, a vacuum system for providing a sub-ambient pressure to a target volume is provided, comprising:
a suction head having an inlet arrangement adapted for being in fluid communication with said target volume;
a vacuum pump in fluid communication with said suction head, said vacuum pump being adapted for providing a predetermined working pressure in said system below external ambient pressure of an external environment with respect to said suction head;
a waste container defining a collection volume for collection of materials that may be drained from said target volume in fluid communication with an outlet port of said vacuum pump; and
a passive pressure regulator for maintaining said working pressure, said passive pressure regulation system having an outlet in fluid communication with said suction head upstream of the said waste container, and said passive pressure regulator system having an inlet in selective fluid communication with said collection volume.

The pressure regulator may comprise a venting valve arrangement adapted for enabling ingress of external ambient air into the system responsive to a reduction in said working pressure below a predetermined datum pressure with respect to said external ambient pressure and for discontinuing said ingress when said datum pressure is restored. The venting valve arrangement may comprise an inlet port having a valve seat in selective fluid communication with said ambient air, and a valve seal biased for sealingly closing with respect to said valve seat by means of a resilient element generating a biasing force of magnitude substantially less than and in a direction generally opposed to a pressure-induced force acting on said valve seal when said working pressure is less than said datum pressure. The pump may comprise a pump head reversibly engageable with a powered pump drive unit, in a manual facile manner, and said container is integral with said pump head, i.e., either integrally formed therewith or suitably joined thereto.

According to a further aspect of the invention, the vacuum system comprises a pump, remote from the target volume and which generates a vacuum thereat, wherein changes in the vacuum level may be monitored via indirect methods in which it is not necessary to directly measure the vacuum level. This monitoring allows blockages to be identified in the system, which give rise to a high vacuum level between the blockage and the pump, and also allows leaks to be identified in the system, as there is a loss of vacuum.

The suction head may be of any shape, size or form without limitation, typically suitable to the particular target volume that is being suctioned. For example, for external body applications in which the target volume is associated with wounds, burns and the like, the suction head may comprises an enclosure sealable to a perimeter of the wound/burn so as to define a confined volume comprising said target volume. In internal body applications, for example tracheal drainage and the like, the suction head may comprise a drain catheter or other similar arrangement having at least one lumen in fluid communication with said pump, and said inlet arrangement comprises at least one aperture adapted for providing fluid communication between said target volume and said at least one lumen.

The pressure regulation system may comprise a venting valve arrangement adapted for enabling ingress of ambient air into the system responsive to a reduction in said working pressure below a predetermined datum pressure with respect to said external ambient pressure and for discontinuing said ingress when said datum pressure is restored. The venting valve arrangement may comprise an inlet port having a valve seat and in fluid communication with said ambient air, an outlet port in fluid communication with said vacuum system, and a valve seal biased for sealingly closing with respect to said valve seat by means of a resilient element generating a biasing force of magnitude substantially less than and in a direction generally opposed to a pressure-induced force acting on said valve seal when said working pressure is less than said datum pressure.

Optionally, the venting valve arrangement comprises an adjustment mechanism for adjusting said datum pressure. In one form of the valve arrangement, said adjustment mechanism comprises a bias adjusting arrangement for adjusting the magnitude of said biasing force. The resilient element may comprise a compression spring mounted in a suitable housing such as to urge said valve seal towards said valve seat, and said bias adjusting arrangement comprises a compression control mechanism for adjusting the compression of said spring.

The pressure regulation system may be connected to the suction head permitting selective fluid communication between said target volume and said ambient air, enabling ingress of ambient air into said target volume responsive to a reduction in said working pressure below a predetermined datum pressure and discontinuing said ingress when said datum pressure is restored.

In any case, the pressure regulation system may be mounted in a location upstream or distal of the waste container, and may further be located as close as possible to the target volume, proximate to, i.e. just downstream or proximal, or comprised at the suction head. In some embodiments, the pressure regulation system may also be located upstream or distal of the suction head, for example when the suction head comprises a wound enclosure. By the mounting location is meant either the location where the pressure regulation valve is actually located, or, where the pressure regulation system comprises a dedicated conduit downstream thereof to transmit the pressure of the pressure regulation valve to a particular location of the vacuum system, where such a conduit is actually connected to the vacuum system. By “proximate” is meant that the pressure regulation system may be closer to the suction head than to the waste container in terms of fluid flow paths between the three components.

Thus, the passive pressure regulation system is in fluid communication with said suction head upstream of the said waste container.

The pressure regulation system may be connected to said suction head directly or via a suitable conduit. In some embodiments, the pressure regulation system may be in selective fluid communication with an outlet port of said vacuum pump and said external ambient air such as to allow ingress of at least one of fluid from said vacuum pump outlet port and said external ambient air when said working pressure is below said datum pressure.

The embodiments of the passive pressure regulation system of the invention comprising a selective venting valve arrangement thus operate in direct response to a particular change in pressure at or close to the suction head, regardless of the actual volume flow rate of the air ingress through the pressure regulation system, and thus operates to maintain substantially constant the operating pressure in the vacuum system responding almost instantaneously to any departure from the set working pressure.

In yet other embodiments of the invention, the pressure regulation system comprises a venting arrangement adapted for providing, at least during operation of said system, substantially permanent fluid communication between said suction head and at least one of said environment, such as to enable said working pressure to be maintained at the suction head while enabling a desired flow rate of ambient air into the suction head via said venting arrangement, and an irrigation source, to enable irrigation of said target volume with a desired irrigation material. Optionally, the venting arrangement may comprise at least one bleeding orifice comprising an effective flow area compatible with providing said desired flow rate. The bleed hole, or other orifice, creates a predetermined pressure drop across it as external ambient air flows through the bleed holes and towards the pump. The flow level will generate the desired vacuum level across the bleed hole, which when connected to the target volume will provide such vacuum control at the target volume. When air flows through such a bleed hole, which restricts the flow, a pressure differential needs to be generated across such hole to force the flow therethrough. To increase the flow requires an increase in the pressure differential, and by changing or controlling the flow through the bleed holes (by controlling the flow through the pump), the pressure differential can be controlled to any desired level. Since one side of the bleed hole, is at ambient pressure, the generated pressure differential across such bleed hole provides the sub-ambient pressure on the other side of such bleed hole, corresponding to a particular setting of the pump.

In some embodiments, the bleeding orifices may be used for venting the target volume via the suction head, while in other embodiments the bleeding holes may be used solely for preventing blockages and/or removing any blockages (including fluids, coagulates, exudates, etc.) in the conduits between the suction head and the pump. In yet other embodiments, the bleeding holes are used for ventilation of the target volume and for removing blockages from the conduit as well.

In other embodiments of the invention, the system comprises:

a suction head having an inlet arrangement adapted for being in fluid communication with said target volume;

a vacuum pump in fluid communication with said suction head, said vacuum pump being adapted for providing a working pressure in said system below external ambient pressure;

a waste container defining a collection volume for collection of materials that may be drained from said target volume in fluid communication with at least one of said vacuum pump and said inlet arrangement; and

wherein said suction head comprises venting arrangement for enabling the suction head to be vented.

The vent arrangement may comprise, for example, bleeding hole, which renders the wound closure vented or non-airtight, as distinguishable from conventional wound closures. The flow of air from the bleeding hole in the wound closure, in response to the negative pressure created by the vacuum pump, facilitates the removal of exudate, which might otherwise coagulate, dry-up and occlude the tubing as well as controls the vacuum level at the wound enclosure.

According to another aspect of the present invention, there is provided a method for draining an open wound from liquids exuded therefrom. The method includes:
providing an enclosure and sealing it to the wound circumference so as to define a confined volume,
connecting the confined volume to a vacuum pump,
connecting a waste container for collection of drained liquids to the vacuum pump, and
operating said vacuum pump to draw the exuded liquids from the wound.

Optionally, the confined volume is connected to an inlet of the vacuum pump and the waste container is connected to an outlet of the vacuum pump such that the drained liquids flow through said vacuum pump.

The method may include employment of an enclosure with bleeding orifices so that ambient air is allowed to enter the tube and flow together with the drained exuded liquids.

Optionally, gases may be separated and released from the drained exuded liquids.

According to a further aspect of the present invention, there is provided a vacuum system for practicing the above method. The vacuum system may use a totally disposable vacuum pump, together with a waste collection bag, for example as disclosed in WO03016719. The vacuum pump may be a two-chambered diaphragm pump adapted for pumping gases and liquids and/or any combination thereof. The vacuum pump may be capable of pumping air and fluid which enter its inlet port, to a waste bag attached to its outlet port. The waste bag may be vented to the atmosphere, such that it collects only the non-gaseous fluids which enter it.

The vacuum pump may comprise a pump head that is releasably operatively coupled to a pump drive unit, wherein said pump head is fixed to or integral with said waste container.

In particular, the pump head and pump drive unit are adapted for enabling quick, easy and simple manual connection and disconnection of the two components, without the need for tools. Thus the pump head and the pump drive unit are attachable and detachable one with respect to the other.

The pump head comprises a pump chamber and a reciprocable pump member that defines part of the pump chamber, and the pump chamber is adapted to expand and contract by two-way forced reciprocation of the pump member under the action of a drive element in the form of a reciprocating member comprised in the pump drive unit. The pump head and the pump drive unit are constructed so that attaching the pump head to the pump drive unit brings the reciprocating pump member and the drive element (reciprocating member) to a position that provides, during operation of the pump drive unit, for their engagement and for reciprocation of the pump member by means of the driving element.

The pump drive unit may comprise first attachment means, and said pump head and/or container may comprise second attachment means, the two attachment means allowing said attaching of the pump head to the pump drive unit by a simple manipulation without tools. Further, the pump head and pump drive unit may be constructed so that said first and second attachment means provide detachment of said drive unit from said pump unit by a manipulation including at the most manual unfastening without tools and one detaching motion, and the same detaching motion disengages said reciprocal pump member from said driving element.

The pump head may comprise a pump inlet port and a pump outlet port, and the pump member may be in the form of a suitable deformable diaphragm arrangement, reciprocable by means of said pump drive unit, to induce said working pressure in said vacuum system. At the same time, the drive unit may comprise a reciprocation drive for driving a reciprocating member, and said diaphragm is releasably engageable with said reciprocating member responsive said pump head being coupled to said pump unit. The reciprocating member and the diaphragm may be conformally shaped such that when coupled one with the other a vacuum is created therebetween as air is expelled, and the vacuum maintains the two components coupled, until a user actively decouples the reciprocating member and the diaphragm one from the other, typically automatically as the pump head is disconnected from the pump drive unit. For example one of the reciprocating member and the diaphragm may comprise a suction cup arrangement for engagement with respect to a substantially smooth surface of the other one of the reciprocating member and the diaphragm. Thus, the attachment of the pump head to the pump drive housing brings the reciprocating elements of the pump drive and the pump head into a position by which the reciprocation of the one will result in the reciprocation of the other. Other arrangements enabling automatic engagement between the pump head and pump unit may be provided.

According to the invention, the pump may be a powered reciprocating pump; the reciprocating member may be in the form of a reciprocating head having a second diaphragm, or, in the form of a piston head arrangement, for example.

A part of the pump head may be accommodated in the waste container, wherein the pump diaphragm is facing a direction generally away from said collection volume, and wherein said pump inlet port and a pump outlet port are at least partially within said collection volume. In one embodiment, the pump inlet port is in fluid communication with said suction head via a conduit that passes through the wall of the container, said pump outlet port is in fluid communication with said collection volume and wherein said waste container is vented to said external ambient air.

In another embodiment, the pump inlet port is in fluid communication with the suction head via said collection volume, and said pump outlet port is vented to said external ambient air. In such an embodiment, the pressure regulation system may be operatively connected to said suction head via a suitable first conduit and wherein said pressure regulation system is in selective fluid communication with said pump outlet port and said external ambient air such as to allow ingress of at least one of fluid from said vacuum pump outlet port and external ambient air into said target volume responsive to a reduction in said working pressure below a predetermined datum pressure and such as to discontinue said ingress when said datum pressure is restored. Further, the waste container may be in fluid communication with said suction head via a suitable second conduit, and optionally the first conduit and the second conduit may be in fluid communication one with another.

At least some, and preferably all of the pump head, waste container, suction head and pressure regulation system are configured for being disposable. Optionally, the pump head and waste container are reversibly lockably engaged with said pump drive unit by means of a latch arrangement.

The target volume may include a medical target volume associated with a wound, burn or the like, and said suction head may comprise an enclosure sealable to a perimeter of the wound burn or the like, respectively, so as to define a confined volume comprising said target volume.

According with an aspect of the present invention, there is provided a disposable assembly for draining an open wound from liquids exuded therefrom. The assembly comprises an enclosure attachable to the wound circumference so as to define a confined volume, a vacuum pump unit connected to the enclosure so that negative pressure may be created in the confined volume, and a waste container connected to the vacuum pump unit. The vacuum pump unit has means for detachably attaching to a drive unit for operating the pump unit. The enclosure is connected to an inlet of the vacuum pump unit and the waste container is connected to an outlet of the vacuum pump unit, such that when the vacuum pump unit is operated the drained liquids flow therethrough.

The pump unit and the drive unit may be adapted for attaching and detaching by simple hand manipulations.

The enclosure may have bleeding holes, for example as described above.

The vacuum pump unit may comprise a two-chambered diaphragm pump adapted for pumping gases and liquids and/or any combination thereof.

The waste container may contain a porous media adapted to soak up the drained liquids and may be in the form of a collapsible or foldable bag.

The drained liquids and air contact only the parts of the disposable assembly. The drained exuded liquids may then be disposed of together with the disposable assembly. More specifically, the pump unit may be disposed of after use, together with the tubing connected to it, as well as the waste bag connected, with its content, and with the wound closure which may be connected to the pump unit via the tube.

According to another aspect of the invention, a vacuum system for providing a sub-ambient pressure to a target volume, comprises:
a suction head having an inlet arrangement adapted for being in fluid communication with said target volume;
a vacuum pump in fluid communication with said suction head, said vacuum pump being adapted for providing a predetermined working pressure in said system below external ambient pressure of an external environment with respect to said suction head;
a first waste container defining a collection volume for collection of materials that may be drained from said target volume, said first waste container being coupled to an upstream part of said vacuum pump; and
a second waste container defining a collection volume for collection of materials that may be drained from said target volume, said second waste container being coupled to a downstream part of said vacuum pump.

According to the invention, the waste container and pump may be connected in any serial order with respect to the wound enclosure, for example with the waste container may be distally and/or proximally located (in terms of the fluid flow path from the suction head) from the wound enclosure with respect to the pump.

The present invention also refers to a kit for use with the system of the invention, in particular comprising all the elements of the system other than the pump drive unit. These elements may be disposable, having a relatively low economic cost compared with the economic cost of the pump drive unit for example, or with the economic cost associated with cleaning, sterilizing and recycling such components. Thus, such a kit may comprise:

a vacuum pump head adapted for releasable operative connection to a powered pump drive unit, said pump head comprising a pump inlet and a pump outlet for enabling working fluid to be pumped through the pump during operation thereof;

a waste container defining a collection volume for collection of drained materials in fluid communication with at least one of said vacuum pump head;

wherein said vacuum pump head is one of attached to or integral with said waste container such that at least one of said pump inlet and said pump outlet is accommodated in said collection volume.

Such a kit may thus also optionally comprise a suction head and/or a passive pressure regulation system as disclosed herein, in particular at least one of the following:
a suction head having an inlet arrangement adapted for being in fluid communication with said target volume;
a suction head comprising an enclosure sealable to a perimeter of a wound so as to define a confined volume comprising said target volume, said enclosure being in fluid communication with at least one of said waste container and said pump inlet;
a passive pressure regulation system for regulating said working pressure.

The present invention also relates to a vacuum system comprising all the elements of a kit as disclosed herein and a pump drive unit, wherein the vacuum pump is in fluid communication with said suction head, the vacuum pump being adapted for providing a working pressure in said system below external ambient pressure, said vacuum pump comprising said pump head releasably operatively connected to said pump drive unit, wherein said pump head comprises a pump inlet port, a pump outlet port and a suitable deformable diaphragm arrangement reciprocable by means of said pump drive unit to induce said working pressure in said vacuum system.

The present invention is also directed to a method for providing a sub-ambient pressure to a target volume or for draining fluids therefrom, comprising:

(a) inducing a working pressure in said target volume below external ambient pressure;

(b) providing a collection volume for collection of drained materials from said target volume; and

(c) regulating the working pressure in said target volume to maintain a positive pressure gradient between said target volume and said collection volume such as to aid flow of said materials from said target volume to said collection volume.

Optionally, step (c) comprises allowing ingress of external ambient air at or near to the target volume responsive to a reduction in said working pressure below a predetermined datum pressure with respect to said external ambient pressure and discontinuing said ingress when said datum pressure is restored. Further optionally, the method may comprise allowing recirculation of fluid, in particular air, between said collection volume and said target volume, or proximal thereto, together with said ingress of external ambient air.

Optionally, step (c) comprises selectively allowing substantially continuous ingress of external ambient air to the target volume, such as to enable said working pressure to be maintained at the target volume while enabling a desired flow rate of ambient air into the target volume.

Optionally, the method comprises the step of providing a pulsating pressure to said target volume by alternately inducing said working pressure at said target volume and alternately venting said target volume to external ambient pressure.

Thus, according to the invention, a suction head in fluid communication with a pump head provides a sub-ambient working pressure to a medical site, enabling drainage thereof to a waste container and/or enhancing healing at the site. A passive pressure regulator enables the working pressure to be maintained at a desired level independent of the fluid flow through the system, while the venting arrangement is dependent on preset controlled flow.

The vacuum system may be adapted to be carried by an ambulatory patient.

Some features of the invention include the following. The adjustable pressure regulator or relief valve may be integrated with the wound enclosure or in fluid connection therewith via a tube or conduit, such that the vacuum conditions or negative or sub-ambient pressure applied at the wound may be accurately controlled thereby, in a passive manner, irrespective of the pump flow or exudates flowing from the wound in the direction of the pump. When the negative pressure is controlled at one end of a tube within which exudates are being moved by the force of such negative pressure, a pressure differential is created across the exudates, which moves it. This pressure differential alters the regulator set pressure. In the present invention, the regulator pressure may be transferred directly to the wound by way of a conduit substantially free of exudates, thus controlling the negative pressure at the point of suction, accurately.

When the sub-ambient pressure reaches the set level of the pressure regulator, the regulator opens to introduce airflow in the general direction of the wound so as to maintain the pre-set sub-ambient pressure level. The flow of air from the pressure regulator towards the general direction of the wound substantially prevents exudates or other fluids from the wound from entering the pressure regulator, or where appropriate, the tube or conduit between the regulator and the wound. The flow of air through the pressure regulator is generally continuous and enables the system to operate at a set vacuum level independent of the actual air flow, and renders the wound enclosure vented or non-airtight, as distinguishable from conventional non-vented wound closures, as well as from wound enclosures that are vented by means of a vent opening, which renders the vacuum conditions dependent on the flow through the vent.

The pump flow can be low at all negative pressure settings of the regulator (also referred to herein as a relief valve), since the set negative pressure of the regulator is obtained almost instantaneously as soon as the regulator opens to ambient, regardless of the actual flow, providing for low pump flow, low energy consumption as well as substantially silent or low-noise (acoustic) operation. The described method of controlling the negative pressure at a location distant from the pump which generates such negative pressure may be employed for various applications, such as medical suctioning. In most medical applications, it may be desired to maintain suctioning within predetermined limits to avoid tissue damage that may occur at high suctioning.

According to another aspect of the present invention, there is provided a waste collection canister or container, integral with the negative pressure regulating valve, whereby the air entering the relief valve is the air pumped to generate the negative pressure, thus circulation of the pump flow in a closed loop prevents the contaminated pumped air from being discharged to the atmosphere, as is common with other negative pressure systems.

According to a further aspect of the present invention, there is provided a vacuum system for practicing the above method. The vacuum system may use a totally disposable vacuum pump, whereby the pump is integrated into the waste canister, such that attaching the pump to its drive will simultaneously attach the waste canister. The integration of the pump into the waste canister eliminates any conduit between the pump and the waste canister, providing for ease of disposability, as well as reducing the noise level generated by the pump which is separated from its surrounding by the waste canister, doubling as a sound barrier.

According to another aspect of the present invention, there is provided a disposable pump with a diaphragm with its outer surface acting as a suction cup, for the purpose of engaging the pump to its drive by having suction coupling when the drive contacts the diaphragm.

Another feature of at least some embodiments of the invention is that the pump drive unit operates the vacuum pump in a reciprocating manner that may induce flow or pressure pulsations to the wound area, which in turn may enhance wound drainage. This pulsation effect may be enhanced in embodiments where the pump inlet is connected directly to the wound enclosure, rather than via a waste canister.

Another feature of at least some embodiments of the invention is that the selective venting effect provided by the pressure regulator serves to vent the wound enclosure when the pressure therein has dropped below a threshold level, allowing relative quick movement of exudate entering the conduit from the enclosure, into the waste collection container before the exudates dries or coagulates and occludes the tube.

Another feature of at least some embodiments of the invention is that the integral unit, comprising the pump head and waste container, optionally together with the wound enclosure and conduit(s) may be easily disconnected from the pump drive unit and disposed of after use, providing an alternative economical and medical solution to that of decontaminating pump parts of the prior art.

Yet another feature of at least some embodiments of the invention is that it can provide a generally reduced operating noise level as compared with the operation of prior art devices. For example, in the embodiments described herein, the pump head is accommodated within the waste container, which dampens any noise generated by the pump drive unit. Moreover, in embodiments where the waste container is in fluid communication with the wound enclosure via the pump head, only a small volume of air needs to be removed from the wound enclosure to achieve the required vacuum conditions. In such embodiments, the pump may operate at relatively low speed, required for relatively low flow rates, which has a corresponding low noise benefit.

A feature of some embodiments is that by having the pressure regulator housed within a sleeve in the waste container, it is possible to recirculate the pumped air from the wound back into the system via the regulator, reducing the possibility of exhausting contaminated air back into the environment, and also facilitates the disposal of the waste container and disposable peripherals such as the tubing, regulator and wound enclosure.

According to another aspect of the invention, a vacuum system is provided comprising a vacuum pump adapted for providing a predetermined working pressure in said system below a reference pressure, said vacuum pump comprising a pump head operatively coupled to a powered pump drive unit, and further comprising a non fluid invasive monitoring system for monitoring said working pressure, said monitoring system comprising at least one sensor for monitoring a working parameter of said powered pump and a comparator unit for comparing monitored data corresponding to said working parameter with threshold data, wherein the or each said working parameter is directly related to a magnitude of said working pressure provided by said vacuum pump. By “non-fluid invasive monitoring” is meant that the said monitoring is done in such a way as to prevent any fluid communication between the fluid in the suction system, the pressure of which it is desired to determine, and the monitoring means, and thus excludes any sensors that are exposed to, and/or directly measure, the fluid pressure.

The monitoring system may comprise a suitable alarm configured for being activated when a magnitude of said monitored data exceeds or is below a magnitude of said threshold data. The monitoring system may comprise a display for displaying said monitored data in any suitable manner. The drive unit comprises an electric motor, and at least one said parameter comprises any one of: motor current, motor voltage, motor power, motor rotational speed, motor torque.

Conversely, when a leak in the system reduces the load on the pump, as it is then operating at a lower vacuum level, such reduced load of the operating pump can be directly related to a leak, without having to make any direct measurement of the vacuum level at any point. Motor current could be one parameter which is monitored and is directly related to pump load/vacuum level. Accordingly, a control system monitors the motor current when the pump is operating properly and generating the proper vacuum level, such that any deviation from the recorded reference would be indicative of either excess load on the pump—occlusion or for reduced pump load—leakage.

Thus, a method is provided for indirectly monitoring a working pressure generated by a vacuum pump in a vacuum system, said working pressure being below a reference pressure, said vacuum pump comprising a pump head operatively coupled to a powered pump drive unit, the method comprising monitoring a working parameter of said powered pump and comparing monitored data corresponding to said working parameter with threshold data, wherein the or each said working parameter is directly related to a magnitude of said working pressure provided by said vacuum pump.

The method may further comprise activating an alarm when a magnitude of said monitored data exceeds or is below a magnitude of said threshold data.

The method may further comprise displaying said monitored data in any suitable manner.

At least one said parameter may comprise any one of: motor current, motor voltage, motor power, motor torque.

Thus, in accordance with this aspect of the present invention, there is provided a vacuum system as described above where the vacuum pump has a drive unit and a control block adapted to power the drive unit so that a predetermined level of negative pressure is maintained in the confined volume. The control block has a sensor for sensing working parameters of the drive unit and means for deriving the level of negative pressure in the confined volume from these working parameters, in order to maintain said predetermined level. The sensor has no fluid connection with the confined volume. For example, the drive unit may comprise a direct current electric motor and the sensor may sense the electric current driving the motor. The same function of negative pressure control may be accomplished by an adjustable torque limiting clutch, placed between the motor output shaft and the pump. The control block may be provided with alarm means to warn the user if the predetermined level of negative pressure is not maintained.

If the vacuum pump comprises a disposable pump unit and the drive unit is detachably attachable to the pump unit, the control block with monitoring means is preferably associated with the drive unit which is non-disposable.

Thus, indirect means are provided for controlling or monitoring the level of negative pressure applied to the wound, without making any direct connection to a vacuum sensor, transducer or gage to any portion of the system, which has the negative pressure applied to it. The indirect negative pressure monitoring and control result from the need to dispose of any portion of the system, which may come in contact with the pumped media, which is likely to be contaminated or infectious. Accordingly, all the disposable components in the system may be relatively low in cost, to promote discarding them after use. Pressure transducers, vacuum gages or sensors, are relatively costly, and thus not generally considered disposable.

### BRIEF DESCRIPTION OF THE DRAWINGS

In order to understand the invention and to see how it may be carried out in practice, a number of embodiments will now be described, by way of non-limiting example only, with reference to the accompanying drawings, in which:

FIG. 1 is a schematic illustration in cross-sectional side view of a first embodiment of the invention, wherein the pump-head/container unit is detached from the pump drive unit, and the wound enclosure is fixed to a wound site.

FIG. 2 is a schematic illustration in cross-sectional side view of a second embodiment of the invention, wherein the pump-head/container unit is attached to the pump drive unit, and the wound enclosure is fixed to a wound site.

FIG. 3 is a schematic illustration in fragmented cross-sectional side view of the pressure regulator of the embodiment of FIG. 2.

FIG. 4 is a schematic illustration in cross-sectional side view of a third embodiment of the invention, wherein the pump-head/container unit is attached to the pump drive unit, and the wound enclosure is fixed to a wound site.

FIG. 5 is a schematic illustration in cross-sectional side view of a fourth embodiment of the invention, wherein the pump-head/container unit is attached to the pump drive unit, and the wound enclosure is fixed to a wound site.

FIG. 6 is a schematic illustration in cross-sectional side view of a variation the pump-head/container unit of the embodiment of FIG. 5 in isolation from the pump drive unit.

FIG. 7 is a schematic illustration in cross-sectional side view of another variation the pump-head/container unit of the embodiment of FIG. 6 in isolation from the pump drive unit.

### DETAILED DESCRIPTION OF EMBODIMENTS

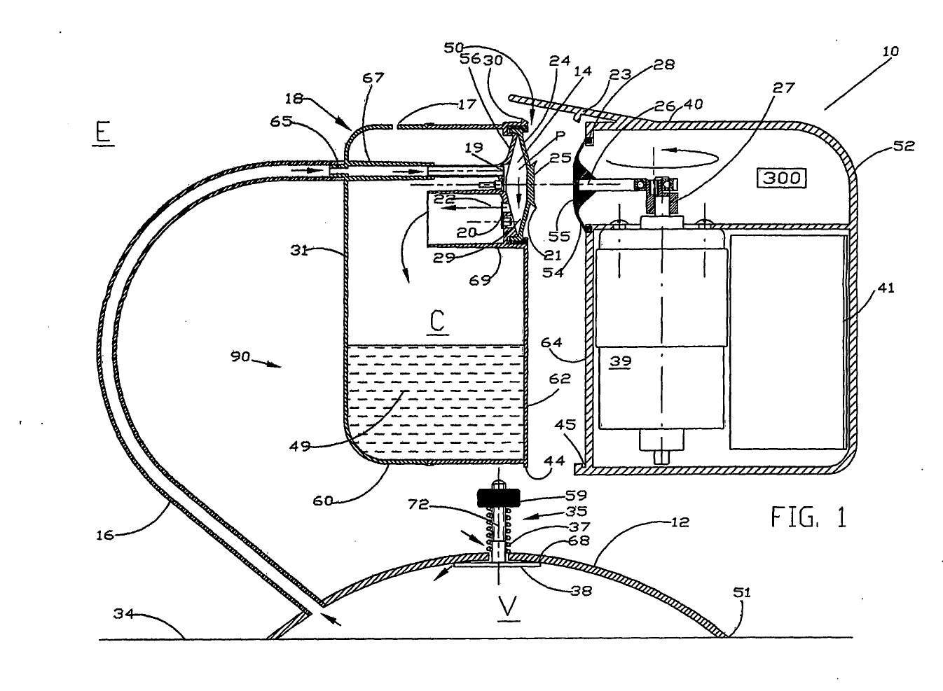

A vacuum system for providing, i.e. applying, a sub-ambient pressure to a medical target volume, and thus for enabling fluids to be drained therefrom according to a first embodiment of the present invention, illustrated in FIG. 1 and generally designated with the numeral 10, comprises a suction head, a vacuum pump 50, waste canister or container 31, and pressure regulator 35.

The suction head has an inlet arrangement adapted for being in fluid communication with the target volume, and may be in the form of wound enclosure 12, which is associated with the target volume comprising a wound, for example. The enclosure 12 has an outer perimeter 51 that is sealable to the periphery of the wound area on the body 34. The enclosure 12 defines a confined volume V including the target volume over the exposed parts of the wound from which it is desired to remove under suction liquids and other flowable materials, which may include biological or non-biological materials, though at times it may be desired merely to maintain a negative pressure in the confined volume V over the wound to promote healing thereof.

In other embodiments, the suction head may instead comprise, for example, a drain catheter or the like, for applying a predetermined vacuum to a medical target area, such as for example the intra-oral cavity, trachea, an organ of the body being operated on, and so on. Such a drain catheter may comprise at least one lumen in fluid communication with the pump 50, and the inlet arrangement may comprise at least one aperture adapted for providing fluid communication between the target volume and the at least one lumen.

The pump 50 is a powered pump, and comprises a pump head 14 that is releasably coupled to a pump drive unit 40 for operation therewith. The drive unit 40 comprises a housing 52 accommodating a powered drive, such as an electric motor 39, and a battery pack 41 for powering the motor. Additionally or alternatively, the motor 39 may be provided with power form an external source, such as for example an electric mains (not shown).

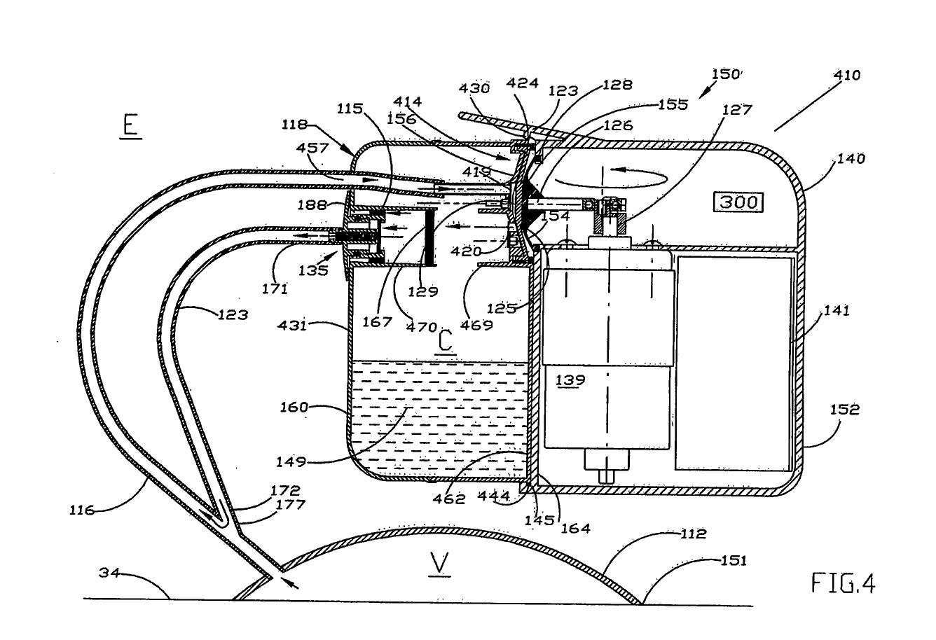

The system 10 may further comprise a system 300 for monitoring and optionally controlling at least one operative parameter of the motor 39, and while this system will be described in greater detail hereinafter with respect to the embodiment of FIG. 4, it applies, mutatis mutandis, to the other embodiments of the invention.

A reciprocating mechanism 27, comprising a crank and a reciprocating head 26 coupled to a rod, is provided within the housing 52 for converting the rotary drive of the motor 39 to reciprocating motion for reciprocating head 26. The reciprocating head 26 comprises a flexible membrane 54 having a periphery 28 thereof suitably sealingly anchored to a frame in said housing 52, such that a relatively smooth surface 55 of the membrane 54 is exposed with respect to the housing 52, and such as to prevent contamination of the inside of the housing 52.

The pump head 14 comprises a pump inlet port 19 and a pump outlet port 20 comprised on a substantially rigid part 56 of the pump head 14, and a flexible diaphragm 24 that is connected at its periphery to the periphery of the rigid part 56 to define a pump working chamber 29 having a variable pump volume P. Suitable one-way valves are provided at the pump inlet port 19 and a pump outlet port 20 to ensure fluid flow in one direction through the pump head 14 from inlet port 19 to outlet port 20. The flexible diaphragm 24 is deformable from a first position in close proximity to the rigid part 56 defining a minimum pump volume Pₘᵢₙ, and a second position (when maximally spaced from the rigid part 56 during operation of the system) defining a maximum pump volume Pₘₐₓ. An outer-facing side of the diaphragm 24 comprises a suction cup 25, integrally or otherwise joined thereto, adapted for releasbly engaging with respect to said smooth surface 55. Optionally, and as illustrated in FIG. 1, the suction cup 25 may comprise a peripheral lip 21.

The waste container 31 comprises a suitable housing 60 defining a collection volume C adapted for collecting waste materials, particularly liquids and other flowable materials, from the wound or other target volume to which the system is coupled. Thus, the housing 60 is substantially at least one of impermeable, contamination and leak-free regarding these materials with respect to the external environment E, and may be formed as an integral item, or from several parts suitably joined together, for example. The container 31 is rigid or semi rigid, though in other variations of the embodiment, the container may be non-rigid, and is suitably adapted for connection with respect to the drive unit 40.

The pump head 14 is joined to said waste container 31, such as to form an integral pump-head/container unit 18. However, the pump head 14 or parts thereof may be formed integrally with the waste container 31, or alternatively each component may be formed separately and joined together in any suitable manner, for example bonding, welding, fastening, and so on, to form the integral unit 18. At least a part of the pump head 14 may be accommodated in the collection volume C, in particular, the pump inlet port 19 and pump outlet port 20 are at least partially accommodated within said collection volume C, while the diaphragm 24 is facing in a direction generally away from collection volume C. The pump head 14 is located with respect to the container 31 at a position such that when the container 31 is coupled to the drive unit 40, the diaphragm 24 is aligned with the smooth surface 55. The natural resilience of the diaphragm 24 generally results in the pump head 14 defining size of variable internal volume P intermediate between the said maximum and minimum pump volume P, if not the maximum pump volume Pₘₐₓ, when the pump head 14 is disengaged from the drive unit 40. In such a position, or even if the diaphragm 24 were to be at the first position defining a minimum pump volume P, the suction pump 25 engages the smooth surface 55 automatically, either immediately when the waste container 31 is coupled to the drive unit 40, or very soon after operation of the drive unit, when the reciprocating mechanism pushes the reciprocating head into engaging contact with the diaphragm 24.

In the first embodiment illustrated in FIG. 1, the enclosure 12 is in direct fluid communication with the pump inlet port 19 via conduit 16 that extends from the enclosure 12 and is connected to a nipple 65 on the container housing 60, and thence via a second conduit 67 that projects into the volume C from the nipple 65 and is sealingly fixed to the inlet port 19.

The outlet port 20 discharges or drains, via optional sleeve 69, fluids that are sucked into the pump head 14 from the enclosure 12 into the collection volume C of the container 31.

In this embodiment, the container 31 also comprises a vent 17 for venting the collection volume C to the external environment E. A suitable biological filter, hydrophobic filter or other filter (not shown) may optionally be provided at vent 17 to prevent contamination of the external environment E from the contents of the container 31.

The container 31, or indeed pump-head/container unit 18, comprises an interface 62 that faces, and has a form that is generally complementary to, a drive unit interface 64 on the housing 52, for facilitating mounting or attaching the pump-head/container unit 18 (or container 31) with respect to the drive unit 40. The system further comprises a suitable coupling and locking mechanism, including suitable attachment arrangements on each of the pump-head/container unit 18 (or container 31) and the housing 52 (or drive unit 40), for allowing attachment or detachment one from the other, and the attachment arrangements allow attaching of the container and/or pump head to the drive unit by a simple manipulation without tools. Such attachment means may comprise, for example latch 23 engageable with tooth 30, and tab 44 engageable with slot 45, which enabling coupling and decoupling of the pump-head/container unit 18 (or container 31) with respect to the drive unit 40.

In this embodiment, the pressure regulator 35 may be mounted to the enclosure 12 at any suitable position, though typically at a position where operation thereof will be unhindered by other equipment or parts of the patient's body, or where operation thereof will not be compromised by specific treatments that may be needed to be administered to the patient. Alternatively, the regulator 35 may be mounted in a suitable conduit, for example at a first end of a conduit that has its second end fixed to, and in open communication with, the enclosure 12, and such an arrangement may be useful in cases where the area directly over the enclosure is unsuitable, for example where the patient is covered with blankets which are also draped over the enclosure.

The pressure regulator 35 comprises a vent valve arrangement having a valve seal 38 that cooperates with valve seat 68 for providing sealing engagement therewith when the regulator 35 is in the closed position. The valve seal 38 is mounted on a pin 72 having a nut 59 thereon, the axial position of which relative to the pin is adjustable. The pin 72 is reciprocably movable within the lumen of a helical spring 37, located between the nut and the enclosure 12, between an open position in which the seal 38 is displaced from the seat 68, and the said closed position. The regulator 35 is urged to the open position when there is a pressure difference between the ambient air pressure of the external ambient environment E and the pressure within the confined volume V that exceeds a threshold value M. When this pressure difference is at or less than the threshold value M, the regulator 35 is urged to the closed position by means of the restoration force of the spring 37. The datum restoration force provided by spring 37 can be adjusted by means of nut 59, in order to control the threshold value M, and thus the vacuum conditions in the confined volume V at which the regulator 35 opens to the external environment E.

Optionally, the regulator 35 may also comprise a biological or other suitable filter to prevent possible contamination of the wound via the confined volume V, and/or possible contamination of the external environment E.

The integral unit 18 comprising container 31 and valve head 14 may be provided as a kit 90, which may also comprise conduit 16, enclosure 12 and regulator 35, optionally already connected to the unit 18. Alternatively the conduit 16, enclosure 12 and regulator 35, may be provided separately. The kit 90 typically also comprises a sterile bag or other packaging (not shown) that is removed before use, and after a single or one-time use it is disposed of, typically in a contamination-free manner. Thus, the unit 18 may be made from relatively inexpensive materials, compared with, for example, the manufacturing costs of the drive unit 40, and in any case may also be made from medically compatible materials, including suitable plastics and so on.

Thus, according to one aspect of the invention, the system comprises a disposable part, including integral unit 18 conduit 16, enclosure 12 and regulator 35, and a reusable part, including the pump drive unit 40.

The system 10 according to the first embodiment may be operated as follows. Unit 18, interconnected with the conduit 16 and enclosure 12, is mounted to drive unit 40, such that the pump head 14 is engaged with the reciprocating head 54, and locked together via latch 23. The control unit 40 may be switched on temporarily for driving the reciprocating head 26 through one or half of a reciprocation cycle as necessary to ensure that the suction cup 25 is firmly engaged on the smooth surface 55, or alternatively this engagement step may be carried out when activating the pump after the system 10 is coupled to the target volume. The enclosure 12 is placed over the wound site so as to cover the same, and the periphery 51 sealingly abutting the body 34, for example with the aid of bandages, dressings, adhesive tape, and so on. The nut 59 is adjusted to provide the required setting for the pressure regulator 35. The drive unit 40 is switched on, and as the motor 39 is activated, the crank turns, reciprocating the rod and reciprocating head 26, causing the diaphragm 24 to reciprocate with diaphragm 54 and thus alternately increase and decreased the pump volume P. Thus, as the pump head 14 begins to operate, air and fluids exuded from the wound are sucked out of the contained volume V, providing a negative pressure thereat and creating a partial vacuum. Fluids and other exudate materials in the wound are drawn and carried through the conduit 12 and conduit 67 directly to the inlet port 19, through the pump working chamber 29 (which is at a below-ambient, or negative, pressure when operating), and out of the outlet port 20 to the container volume C via discharge sleeve 69. If the pressure in the confined volume V drops too much, then the pressure difference threshold M is exceeded, and the seal 38 becomes unseated, allowing ingress of external ambient air into the confined volume V. The flow of air into the confined volume V vents the enclosure and aids in entraining the fluids and materials from the wound towards the pump head 14, in particular where they may be blocking part of the passage to the inlet port 19. As diaphragm 24 reciprocates, it may induce partial cyclic flow within the conduit 16 as the air pulsates, particularly where the connection between the pump head 14 and the enclosure 12 is short, and this may cause the pressure in the enclosure 12, and thus the pressure to the wound area on the body 34 to pulsate accordingly, i.e., to fluctuate to some degree, enhancing drainage of exudates from the wound and/or enhancing the healing process by massaging this area. The relatively sudden opening of the pressure regulator 35, under the appropriate pressure conditions, may also provide a pulse effect that may help to dislodge blockages etc.

If necessary, the nut 59 may be adjusted to allow operation of the regulation valve at lower or higher vacuum levels in the enclosed volume V. As exudates fills the collection volume C, air is displaced out of this volume via vent 17.

If conduit 16 becomes blocked, the vacuum created by the pump 50 is increased by the action of the pump head 14 until the blockage is dislodged and displaced to the waste container, which may have the effect of reducing the vacuum in the contained volume V, which in turn may cause the regulator to open and allow air thereinto.

If the container volume C of container 31 reaches full capacity, for example the collected materials or exudates 49 reaching the level of the outlet port 20 or any other suitable level, the unit 18, conduit 16, and enclosure 12 may be disconnected from the drive unit 40 and disposed of, in a similar manner to an end of treatment scenario, as described below, and a new unit 18, conduit 16, and enclosure 12 used with the drive unit 40 to continue treatment, the pump unit having been switched off while the switching of disposable components is taking place. Alternatively, it is possible to remove and dispose of the unit 18, optionally including conduit 16, and to replace just these items to continue treatment. In such a case, patient discomfort is reduced, as the wound site is left alone. In other situations it may be necessary to change or replace the wound enclosure 12 while leaving the conduit 16 and/or the unit 18 in place. Thus, sometimes a kit comprising the range of items including unit 18, conduit 16, and enclosure 12 is useful, while at other times a variety of kits comprising just unit 18, or unit 18 and conduit 16, or conduit 16, or conduit 16 and enclosure 12, or enclosure 12 may also be useful.

After the completion of the wound suction treatment, the drive unit 40 is switched off, and the unit 18 is unlatched from the drive unit 40, automatically disengaging the pump head 14 from the reciprocating head 26, and the wound enclosure is removed from the patient. The unit 18, conduit 16, and enclosure 12 are then disposed of.

Thus, once set, the system 10 effectively provides a desired vacuum level at the wound enclosure 12, which may be remote from the pump head 14, and these conditions may be substantially maintained without having to continually directly monitor the vacuum level at the wound enclosure 12 and to adjust the vacuum generated by the pump head 14 to compensate for changes thereof via a closed loop type control system.

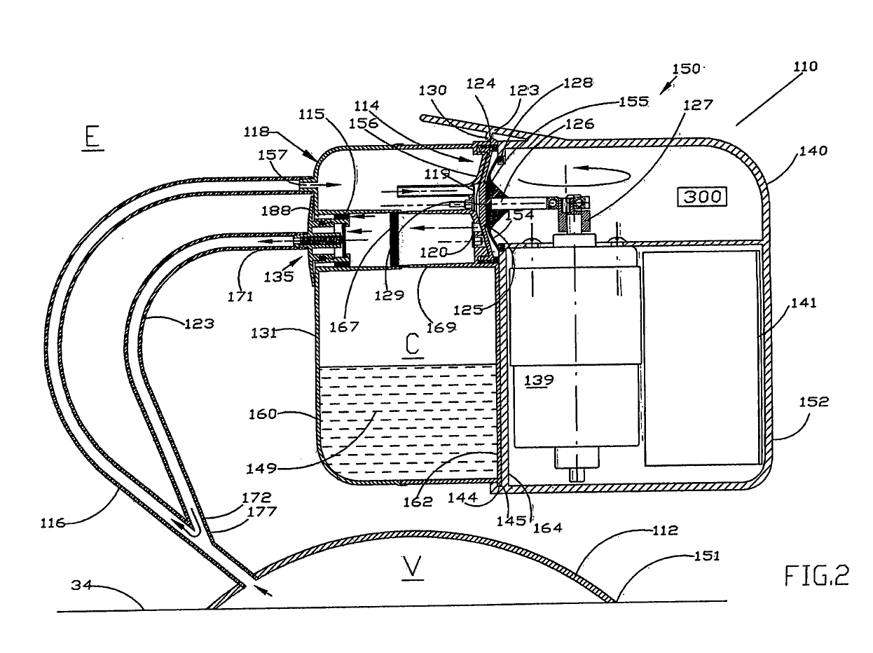

A vacuum system for draining an open wound according to a second embodiment of the present invention, illustrated in FIG. 2, comprises the elements and features of the first embodiment, with some differences as described below, mutatis mutandis. Thus, the system 110 according to the second embodiment also comprises a wound enclosure 112, and a vacuum pump 150, waste canister or container 131, and pressure regulator 135.

The wound enclosure 112 is similar to that of the first embodiment, mutatis mutandis, having an outer perimeter 151 that is sealable to the periphery of the wound area on the body 34 and defining a confined volume V, with one difference with respect to the first embodiment being that the pressure regulator 135 is not mounted to the enclosure 112, but elsewhere as will be further explained.

The vacuum pump 150 is similar to that of the first embodiment, mutatis mutandis, and thus comprises a pump head 114 that is releasably coupled to a pump drive unit 140 for operation therewith, the drive unit 140 comprising housing 152, drive unit interface 164, electric motor 139, battery pack 141, a system 300 for monitoring and optionally controlling at least one operative parameter of the motor 139, reciprocating mechanism 127 (comprising a crank, rod, reciprocating head 126 having a flexible membrane 154 comprising periphery 128, and smooth surface 155, similar to the corresponding components of the pump drive unit 40 described for the first embodiment, mutatis mutandis. Thus, the pump head 114 may be used with the pump drive unit 40 of the first embodiment, and the pump head 14 of the first embodiment may be used with the pump drive unit 140 of the second embodiment.

The pump head 114 comprises rigid part 156 having a pump inlet port 119 and a pump outlet port 120 with suitable one-way valves, and a flexible diaphragm 124 (having a suction cup 125), defining a pump working chamber 129 having a variable pump volume P, similar to the corresponding components described for the first embodiment, mutatis mutandis.

The waste container 131 is similar to that of the first embodiment, mutatis mutandis, and thus comprises housing 160 defining collection volume C, interface 162, coupling/decoupling and locking mechanisms, for example such as latch 123 and tooth 130, tab 144 and slot 145, similar to the corresponding components described for the first embodiment, mutatis mutandis.

In the second embodiment, the pump head 114 is also joined to said waste container 131, to form an integral unit 118 similar to the corresponding components described for the first embodiment, mutatis mutandis, and the pump inlet port 119 and pump outlet port 120 are at least partially accommodated within said collection volume C, while the diaphragm 124 is facing in a direction generally away therefrom.

In contrast with the first embodiment, in the second embodiment the enclosure 112 is in direct fluid communication, via conduit 116 and waste container inlet port defined by nipple 157, with the container 131, rather than the pump inlet port 19, which in this embodiment opens to the collection volume C. Thus, exudates from the wound are directly discharged to the collection volume C. The outlet port 120, on the other hand, discharges to sleeve 169 that extends to the outside of the housing 160 via exit port 188. A suitable filter 167 may optionally be provided between the outlet port 120 and exit port 188 to prevent contamination. The outlet port 120 is thus vented to the external ambient environment E, and thus there is no direct communication between the container volume C and the outlet port 120. Optionally, a baffle plate arrangement (not shown) may be provided downstream of the outlet port 120, which may be useful in attenuating noise with respect to the external environment.

The container 131 does not comprise a vent corresponding to vent 17 of the first embodiment for venting the collection volume C. In the second embodiment, the pressure regulator 135 is mounted to a first end 171 of a second conduit 123 that provides fluid communication between the pressure regulator 135 and the enclosure 112. The second end 172 of conduit 123 is spliced from conduit 116 at juncture 177, so that there is a tube conduit connected to enclosure 112, bifurcating to conduits 116 and 123. Juncture 177 is close to the enclosure 112, but alternatively may be at any other location along the length of conduit 116. Alternatively, in other variations of this embodiment, the second conduit 123 may be connected directly to the enclosure independently of the first conduit 116. Alternatively, in yet other variations of this embodiment, the second conduit 123 may be connected to the first conduit 116 along a length thereof, and optionally may comprise separate lumens of a bi-lumen conduit. Other arrangements for the conduits 123, 116 are also possible.

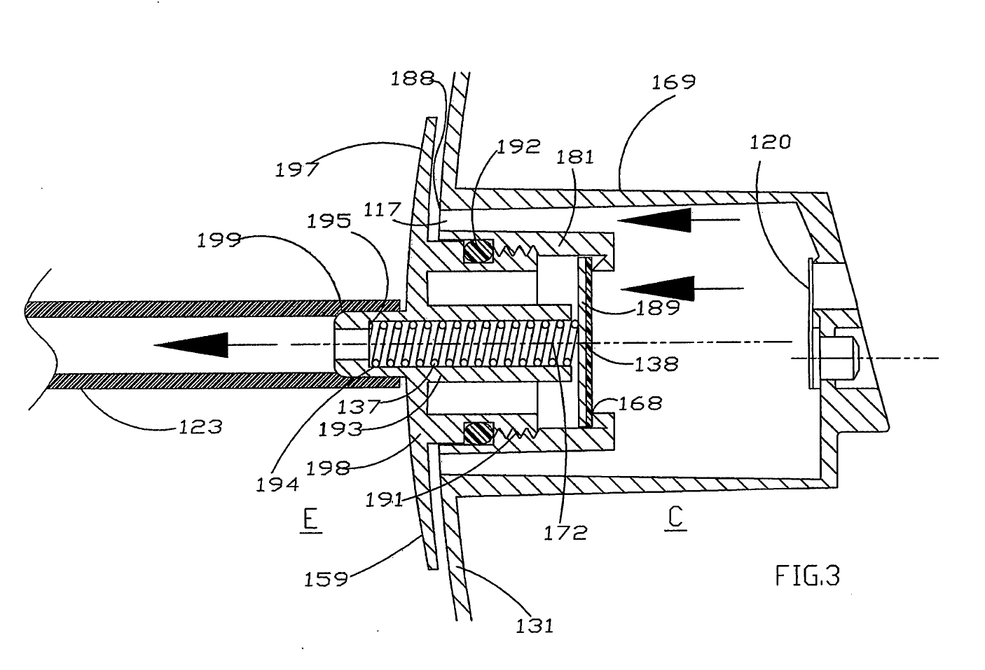

As illustrated in greater detail in FIG. 3, the pressure regulator 135 may be coaxially accommodated in sleeve 169, such as to allow ventilation of the space within sleeve 169 via annular gap 117 formed between.

Optionally, the vent 117 may also comprise a biological or other suitable filter, such as for example a charcoal filter 115 (shown in FIG. 2) to prevent possible contamination of the external environment E and/or transmission of undesirable odors thereto exiting the system via flow from the outlet port 120.

The pressure regulator 135 according to the second embodiment comprises a vent valve arrangement having an outer annular valve body 181 coaxially received within said sleeve 169 and defining said annular gap 117. The valve body inwardly projects from said exit port 188, and comprises a radial inner facing flange defining annular valve seat 168. The valve body 181 is generally static, and may be integrally formed or otherwise joined, mounted or connected in coaxial relationship within sleeve 169. An annular adjustor nut 159 comprises an outer annular body 198 that is adjustably engageable with the valve body 181 via screwthreads 191 that allow controlled relative axial displacement between the nut 159 and valve body 181. Seal 192 prevents leakage between the valve components. The nut 159 comprises an inner sleeve 193 coaxial with and radially displaced with respect to outer annular body 198 via annular plate 197. The inner sleeve 193 defines a stepped lumen 194 with annular shoulder 195 therethrough. Valve seal 138, backed by disc or plate 189, is biased by compression helical spring 137 against valve seat 168. Helical spring 137, which is seated on shoulder 195, is reciprocably movable within the lumen 194 to enable the valve seal 138 to cooperate with valve seat 168 for providing sealing engagement therewith when the regulator 135 is in the closed position, and to enable the valve seal 138 to be displaced from valve seat 168 when the regulator 135 is in the open position. The lumen 194 extends via nipple 199 in a direction towards the outside of the container 131 and allows connection of said end 171 of conduit 123 thereto.

Thus, in the embodiment of FIG. 3, a helical spring 137 can be compressed to a varying degree of adjustment, to provide for a varying level of desired vacuum regulation. In variations of this embodiment, a similar structure for the pressure regulator 135 may be provided, mutatis mutandis, in which the screwthreads 191 are omitted, and wherein helical spring 137 is pre-compressed and non adjustable, to obtain a particular pre-set desirable vacuum level.

In a similar manner to the first embodiment, mutatis mutandis, the regulator 135 is urged to the open position when there is a pressure difference between the ambient air pressure of the external ambient environment E and the pressure within the confined volume V that exceeds a threshold value M. As the sleeve 169 is vented to the external ambient environment E, via vent 117, the ambient pressure of the external ambient environment E is maintained in sleeve 169, and thus with respect to the seal 138 and outlet port 120. When this pressure difference is at, or less than, the threshold value M, the regulator 135 is urged to the closed position by means of the restoration force of the spring 137.

The datum restoration force provided by spring 137 can be adjusted by means of a compression control mechanism in the form of nut 159, in order to control the compression of the spring, and the magnitude of threshold value M, and thus the vacuum conditions in the confined volume V at which the regulator 135 opens to the external environment E. As the nut 159 is rotated clockwise or anticlockwise, the relative axial relationship between the shoulder 195 and valve seat 168 is varied in one or another direction, thereby adjusting the compression force of spring 137 which is applied directly onto the valve seal 138 that is necessary to unseat and open the pressure regulator 135. Optionally, the system 110 may be configured such that the nut is only actuable using a special tool, and thus prevent unauthorized or accidental actuation thereof.

As with the first embodiment, mutatis mutandis, the integral unit 118 may be provided as a kit 90, which may also optionally comprise one or more of conduits 116 and 123, regulator 135 and enclosure 112, optionally already connected to the unit 118, or alternatively, one or more of conduits 116 and 123, regulator 135 and enclosure 112, may be provided separately to the integral unit 118.

The system 110 according to the second embodiment may be operated in a similar manner to the first embodiment. Unit 118, interconnected with the conduits 116, 123 and enclosure 112, is mounted to drive unit 140, and the enclosure 112 is placed over the wound site in a similar manner to that with the first embodiment, mutatis mutandis. The nut 159 is adjusted to provide the required setting for the pressure regulator 135, and the drive unit 140 is switched on. As the pump head 14 begins to operate, air and fluids exuded from the wound are sucked out of the contained volume V, and are drawn and carried through the conduit 112, most of the liquid exudates discharging directly to the collection volume C of the container 131. However air is drawn into the inlet port 119, through the pump working chamber 129 (which is at a below-ambient, or negative, pressure when operating), and out of the outlet port 20 to the sleeve 169, and subsequently vented. If the pressure in the confined volume V drops such that the pressure difference threshold M is exceeded, and the seal 138 becomes unseated, allowing ingress of external ambient air and/or the air discharged from the outlet port 120 into the conduit 123 via sleeve 169. This ingressed air then recirculates to the collection volume C via conduit 116, and this flow recirculation also entrains air and flowable material from the wound enclosure 112. If, in other variations of the second embodiment, the conduit 123 is attached directly to the wound enclosure 112, independently of conduit 116, then the recirculating flow also directly includes the confined volume V as well. In any case, the recirculating flow of air helps to vents the enclosure 112 and aids in entraining the fluids and materials from the wound towards the pump head 114. This recirculation also helps to reduce the amount of contaminated air that may be discharged to the external environment. As with the first embodiment, mutatis mutandis, the nut 159 may be adjusted to allow operation of the regulation valve at lower or higher vacuum levels in the enclosed volume V. On the other hand, the relatively large collection volume C disposed between the wound enclosure 112 and the pump inlet port 119 tends to dampen the pulsating effect generated by the reciprocating operation of the pump head 114.

Disconnection of the unit 118, with or without the conduits 112, 123, pressure regulator 135, and enclosure 112 is similar to that described for the first embodiment, mutates mutandis, and the used components are then disposed of.

A third embodiment of the invention is illustrated in FIG. 4, generally designated 410, comprises combinations of the elements and features of the first and second embodiments, mutatis mutandis, as follows. The system 410 comprises a wound enclosure 112, and a vacuum pump 150 (including pump head 414 and pump drive unit 140), and pressure regulator 135 substantially as described for the second embodiment, mutatis mutandis, including all possible variations thereof, mutatis mutandis. The system 410 also comprises a waste container 431, which is similar to that of the first embodiment, mutatis mutandis, and thus comprises housing 460 defining collection volume C, interface 462, coupling/decoupling and locking mechanisms, for example such as latch 123 and tooth 430, tab 444 and slot 145, similar to the corresponding components described for the first embodiment, mutatis mutandis. As in the second embodiment the enclosure 112 is in direct fluid communication, via conduit 116 and waste container inlet port 457, with the container 431, which in a similar manner to the first embodiment is in fluid communication with pump inlet port 419 of pump head 414.

Thus, exudates from the wound are discharged to the collection volume C via pump head 414 and outlet port 420, in a similar manner to that of the first embodiment, mutatis mutandis. Thus, in contrast with the second embodiment, sleeve 469 does not extend to the outside of the housing 460, but rather is in direct fluid communication with the volume C.

As with the second embodiment, the container 431 comprises sleeve 470 similar to a part of the sleeve 169 of the second embodiment, and the pressure regulator 135 may be coaxially accommodated in sleeve 470, such as to allow ventilation of the space within sleeve 470 via annular gap 117 formed between.

The system 410 according to the third embodiment may be operated in a similar manner to the second embodiment, mutatis mutandis. Unit 418, comprising the container 431 and pump head 414, interconnected with the conduits 116, 123 and enclosure 112, is mounted to drive unit 140, and the enclosure 112 is placed over the wound site in a similar manner to that with the second embodiment, mutatis mutandis. The nut 159 is adjusted to provide the required setting for the pressure regulator 135, and the drive unit 140 is switched on. As the pump head 14 begins to operate, air and fluids exuded from the wound are sucked out of the contained volume V, and are drawn and carried through the conduit 112, most of the liquid exudates discharging to the collection volume C of the container 131 via pump head 414. If the pressure in the confined volume V drops such that the pressure difference threshold M is exceeded, and the seal 138 becomes unseated, allowing ingress of external ambient air and/or the air discharged from the outlet port 420 into the conduit 123 via sleeve 470. This ingressed air then recirculates to the collection volume C via conduit 116, and this flow recirculation also entrains air and flowable material from the wound enclosure 112. If, in other variations of the second embodiment, the conduit 123 is attached directly to the wound enclosure 112, independently of conduit 116, then the recirculating flow also directly includes the confined volume V as well. In any case, the recirculating flow of air helps to vents the enclosure 112 and aids in entraining the fluids and materials from the wound towards the pump head 114. This recirculation also helps to reduce the amount of contaminated air that may be discharged to the external environment. As with the second embodiment, mutatis mutandis, the nut 159 may be adjusted to allow operation of the regulation valve at lower or higher vacuum levels in the enclosed volume V. On the other hand, the relatively large collection volume C disposed between the wound enclosure 112 and the pump inlet port 119 tends to dampen the pulsating effect generated by the reciprocating operation of the pump head 114.

Disconnection of the unit 418, with or without the conduits 112, 123, pressure regulator 135, and enclosure 112 is similar to that described for the second embodiment, mutatis mutandis, and the used components are then disposed of.

In the third embodiment, the pump head 414 may also be joined to said waste container 431, to form an integral unit 118 similar to the corresponding components described for the first embodiment, mutatis mutandis, and the pump inlet port 419 and pump outlet port 420 are at least partially accommodated within said collection volume C, while the diaphragm 424 is facing in a direction generally away therefrom.

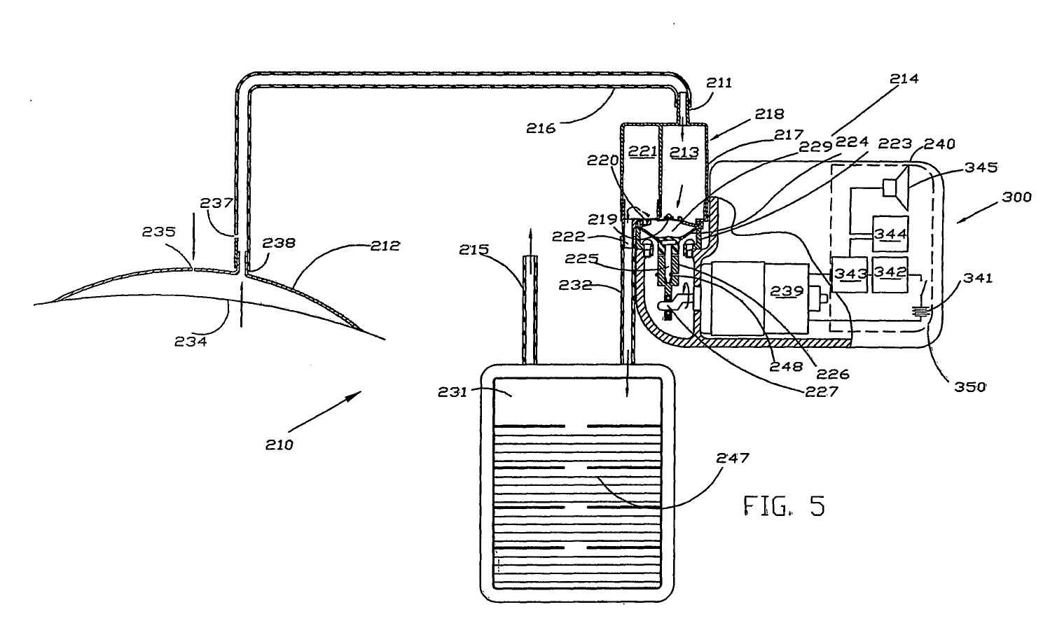

Referring to FIGS. 5, 6 and 7, a vacuum system 210 according to the fourth embodiment of the invention, comprises a wound enclosure 212, a vacuum pump 214, and waste collection canister or container 231, which in the illustrated embodiment is in the form of a flexible container. In the illustrated embodiment, the wound enclosure 212 is particularly adapted for draining an open wound from liquids exuded therefrom, but in other variations of this embodiment the wound enclosure is adapted for any other target volume, for example as described for the first and second embodiments, mutatis mutandis.

The wound enclosure 212 is connected by a suction tube 216 to an inlet 211 of the vacuum pump. The waste collection container 231 is connected to an outlet 222 of the vacuum pump. Thereby, when the vacuum pump 214 is operated, the drained liquids flow through the pump into the waste container 231.

Suction tube 216 is connected to nipple 238 of the enclosure 212 which covers wound area of the body 234 such that suction of air through tube 216 creates negative pressure in the volume above the wound area of the body 234.

A venting arrangement for the wound enclosure 212 is provided, that is configured for allowing a certain degree of venting of the enclosure by ambient air, when the pump 214 is in operation, such as to enable a predetermined vacuum level to be maintained at the wound enclosure 212, concurrent with providing a predetermined throughflow of ambient air into the wound enclosure 212 via the venting arrangement. The venting arrangement may comprise at least one bleeding orifice 235 provided within the enclosure 212, or adjacent to it as a tube orifice 237, allowing ambient air to flow into and through suction tube 216, rendering enclosure 212 non-air tight, or vented.

The vent feature of this embodiment, unlike conventional sealed closures that are not vented, provides for quick movement of exudate entering suction tube 216, toward the vacuum pump 214, and into the waste container 231, before it dries up or coagulates and occludes the tube. This feature also provides for introduction of air at ambient pressure to the wound area of the body 234, and thus equalisation of the air pressure at the wound area with ambient pressure, whenever the vacuum pump stops pumping, allowing cyclic negative pressure application to the wound, by cycling the vacuum pump on and off alternately.

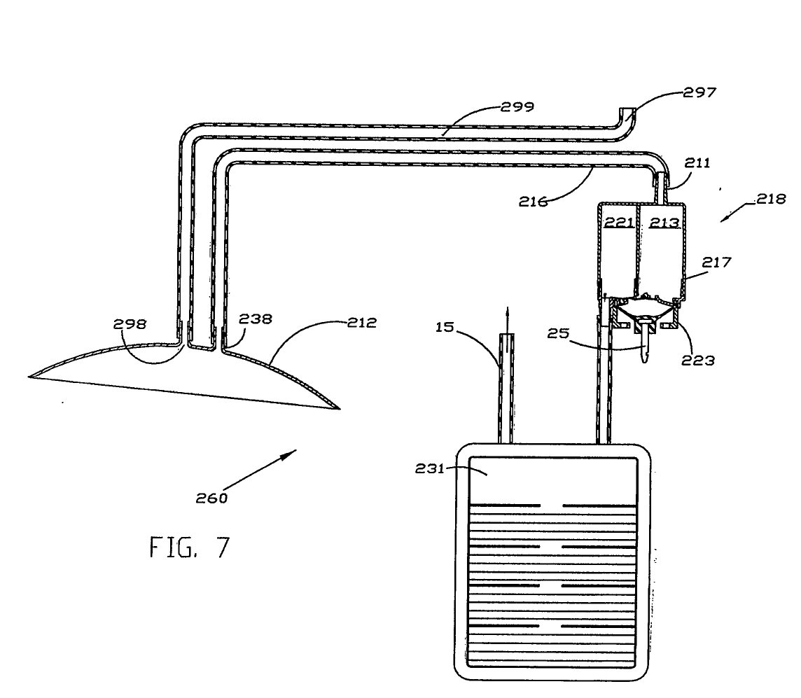

Additionally or alternatively, and as illustrated in FIG. 7, a conduit 299 may be connected to an opening 298 in the enclosure, and used to vent the wound enclosure to a remote location. Optionally, the free end 297 of the conduit may be connected to a suitable pressure regulator, such as for example the pressure regulator described herein with respect to the first or second embodiments, mutatis mutandis, and/or to a suitable irrigation source for introducing irrigation fluids for irrigating and/or sterilizing the body area 234.

Further optionally, openings 298 and 238 may be merged such that conduits 216 and 299 are connected to a single merged opening of the wound enclosure 212 via a Y-connection. In such an arrangement, transportation of exudates etc through the conduit 216 may be enhanced by the air flow originating from the conduit 299, and thus may be further modified so as to maximize movement of such exudates and the like, with minimal or no venting of the volume V itself. A similar effect may be obtained in the embodiment illustrated in FIG. 5, by retaining bleed hole 237 and blocking bleed hole 235. Alternatively, both bleed holes 237 and 235 may be retained, and according to their respective effective flow areas, the proportion of ambient air entering the conduit 216 for the sole purpose of removing coagulate or other exudate, fluids etc therefrom, i.e. entering directly via bleed hole 237, and the proportion of air entering bleed hole 235, and also used for venting the body area 234, can be controlled.

Alternatively of or additionally to the bleeding orifice 235 in the enclosure, a calibrated orifice or other flow restrictors may be used to provide for controlled flow of ambient air into the enclosure or into the outlet. For instance, the wound enclosure may comprise a hole plugged with open cell foam or an open pore sintered metal plug, which restrict the flow, but are not susceptible to plugging, as small dust particles will generally not plug a porous material, unlike a small orifice.

The vacuum pump 214 comprises a pump unit 218 and a drive unit 240 which are detachably attachable to one another, as explained below. The pump unit 18 includes a two-chambered housing 217 and a diaphragm 224 secured to the underside of the two-chambered housing 217 so as to form a working chamber 229.

The two-chambered housing 217 comprises a substantially rigid upstream first chamber 213 with the inlet 211 and a substantially rigid downstream second chamber 221 with an outlet 222. The suction tube 216 is connected to the inlet 211. Two one-way valves 219 and 220 are present at the bottom of the first chamber 213 and the second chamber 221, respectively. To the bottom of the two-chambered housing 217 there is attached a mounting base 223, used to mount the housing 217 to the drive unit 240 by means of a bayonet lock or other suitable manual, facile engagement arrangement.

According to the specific geometry of the first chamber 213 and of the second chamber 221, these chambers may provide an upstream and/or a downstream rigid collection chamber(s) for optionally collecting waste from the volume V, and optionally, the container 213 may be omitted altogether, and thus also function as upstream and/or downstream waste containers, respectively.

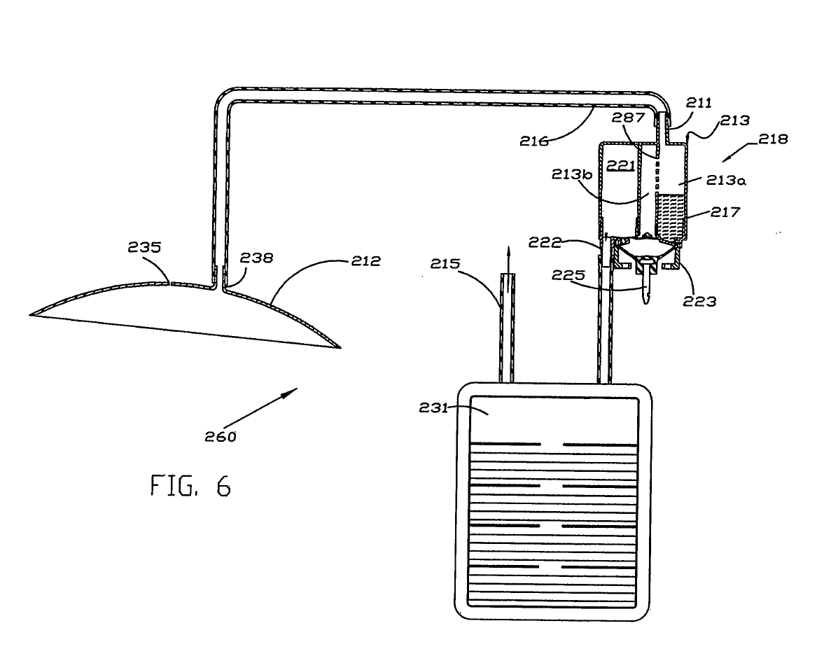

Further optionally, and as illustrated in FIG. 6, for example, the first chamber 213 may be split into two adjacent chambers 213a, 213b, wherein fluids (and possibly solids) are first routed to chamber 213a from the wound enclosure 212, while air passes onto chamber 213b via apertures 287 in the connecting wall between the two adjacent chambers 213a, 213b, When the level of liquid in the chamber 213a reaches the lowermost aperture 287, some of the liquid may begin to overflow into the chamber 213b, and from there to the container via pump chamber 229. In this variation of the fourth embodiment, the system comprises both an upstream collection chamber (chamber 213a), and a downstream collection chamber (container 231). In yet further variations of this embodiment, the upstream collection chamber 213a may optionally be configured, and thus have sufficient volumetric capacity, for example) for collecting substantially all of the liquid (and solids that may be present too) therein that it is to be collected via the wound enclosure, and thus may omit the container 231 altogether.

Similar modifications to the first and second embodiments may be made, mutatis mutandis, to enable these embodiments to collect liquids (and solids) upstream and/or downstream of the pump.

The diaphragm 224 comprises an integral rod-shaped drive member 225, which is used for engagement with the drive unit 240.

The drive unit 240 includes an electric motor 239, batteries 241 and a system 300 for monitoring and optionally controlling at least one operative parameter of the motor 239 described below. The shaft of the motor 239 has a crank 227 coupled to a drive element in the form of a reciprocating rod 226. The rod 226 has a receptacle with a cavity adapted to receive and lock therein the drive member 225.

When the pump unit 218 is attached to the drive unit 240 by means of the bayonet lock in the base 223, the drive member 225 is received in the receptacle cavity of the reciprocating rod 226 to be locked therein upon rotation of the motor shaft 239.

For example, the drive member 225 and the drive element 226 are positioned with respect to the pump unit 218 and the drive unit 240, respectively, for providing mutual alignment between the drive member and said drive element responsive to the pump unit being attached to said drive unit. Furthermore, the drive member 225 and the drive element 226 are configured for mutual engagement thereof, when thus aligned, responsive at least to operation of said drive unit, such as to enable reciprocation of the drive member by means of the driving element during operation of said drive element.

Thus, upon activation of the motor 239, the crank 227 is rotated and reciprocates the receptacle rod 226, causing the diaphragm 224 to expand and contract the working chamber 229. Thus the pump unit 218 pumps air or liquid that passes through the one-way valves 219 and 220.

Air and liquids enter the two-chambered housing 217 through the inlet 211 and suction tube 216 which is connected to the patient's wound enclosure 212, for the removal of exudate. Liquids and air enter the first chamber 213, which is under negative pressure when diaphragm 224 reciprocates, driving them past one-way valve 220, into the second chamber 221. The air and liquid which are pumped through outlet 222, enter waste container 231.

As for the first and second embodiments, the pump's ability to pump air and liquid, unlike conventional pumps, which are efficient in pumping only one type of matter, is enhanced by the flexibility of the diaphragm 224 which optionally allows the diaphragm to yield when encountering heavy loads, such as those present when pumping liquid. This diaphragm flexibility also provides an additional substantial advantage: when the negative pressure in working chamber 229 is high, the diaphragm 224 stretches to allow the reciprocation of the receptacle rod 226 to occur, at minimal burden to the electric motor 239.

The waste container 231 has a vent 215, through which air and gas are discharged to the atmosphere, and optionally a suitable filter may be provided at the vent 215. Accordingly, waste container 231 will retain only the waste fluids which are pumped into it. The waste container may contain a porous media 247 adapted to soak up the drained liquids.

It should be noted that pump unit 218 and the drive unit 240 used in this embodiment, in particular the manner in which the two components are aligned and engaged with one another, may be replaced with a similar structure to that described for the first and second embodiments, mutatis mutandis. Conversely, the pump unit and drive unit of the first and second embodiments could also be replaced with the pump unit 218 and the drive unit 240 of the fourth embodiment, mutatis mutandis.

The pump unit 218 and the drive unit 240 may be calibrated such as to create a performance table or the like, which provides a relationship between operational parameters of the drive unit 240 with respect to negative pressure generated by the pump unit 218. This may be further refined, taking into account a range of effective orifice size, or other parameter that is related to the amount of ventilation provided by the venting arrangement. Thus, once calibrated, whenever a particular negative pressure is required at the wound enclosure 212, for a given venting arrangement ventilation, it may be assumed that this will be provided via a particular pump setting, obtained from the aforesaid tabulated performance values.

Thus, once set, the system 210 effectively provides a desired vacuum level at the wound enclosure 212, which may be remote from the pump head 214, and these conditions may be substantially maintained without having to continually directly monitor the vacuum level at the wound enclosure 212 and to adjust the vacuum generated by the pump head 214 to compensate for changes thereof via a closed loop type control system.

Optionally, the waste container 231 may be made from thin plastic sheet or any other suitable flexible or non-rigid material, which allow it to be folded or collapsed when not full, providing the convenience of having minimal bulk and minimal inconvenience to the patient using or carrying it. Alternatively, the waste container may be made from a rigid or semi rigid material, and may optionally be integrally formed or joined to the pump unit 218.

FIG. 6 shows the disposable portion of the system as an assembly 260, which includes the pump unit 218, the waste container 231, the connecting tube 216 and the wound enclosure 212, all separated from the drive unit and from the wound area of the body 234. As with other embodiments described herein, the assembly 260 may be disposed of in its totality after use, and replaced by a new assembly, thus keeping the costly drive unit 240 free from any contamination. It is clear that the present invention alleviates the need for cleaning or disinfecting any portion of drive unit after use, or providing protective means, such as filters, to keep contaminants from reaching the costly drive.

While in the fourth embodiment, the wound enclosure is freely ventilated, in the first, second and third embodiments, the vacuum level in the wound enclosure may be passively regulated. Thus, in these embodiments, the vacuum level is controlled by an open-loop type technique, by means of setting up a predetermined vacuum level generated by the vacuum head, and this vacuum level may be maintained at the target volume unless substantial leaks and/or blockages occur in the system.

According to another aspect of the invention, a system 300 and corresponding method are provided for monitoring and optionally controlling variations in the sub-ambient pressure level generated by the pump, i.e., without the need for directly measuring air pressure at any point between or beyond the wound enclosure and the pump, minimizing risk of contamination to or from the fluid being pumped therethrough. The system 300 allows for monitoring of the vacuum system of the invention for leaks and/or blockages therein in a non-invasive manner in terms of the pump unit or any of the fluid paths upstream or downstream thereof, and further provides the option of controlling the operation of the drive unit of the vacuum system to compensate for the leakage and/or blockage.

Referring for example to FIG. 5, the drive unit 240 according to the fourth embodiment comprises said system 300, which includes a control block 350 with control circuits such as duty cycle controller 242, which turns the motor pump on and off alternately, motor voltage and current monitoring and controller 243, which controls the negative pressure level produced by the pump unit 218, by controlling the voltage and current which drive motor 239. At any given voltage which drives motor 239, the current draw of the motor is directly related to the negative pressure generated by the pump 218. Accordingly, monitoring of the current which the motor 239 draws provides indirect monitoring of the negative pressure generated by pump 218. The ability to monitor the negative pressure developed by pump 218, indirectly, precludes the need of making an infectious negative pressure line connection to a pressure transducer or vacuum gauge.

For example, if the motor 239 is a direct current electric motor, a sensor, such as for example an ammeter, may measure or otherwise sense the electric current driving the motor. Since the direct current motor output torque is directly related to the current driving the motor, and since the motor output torque is directly related to the negative pressure the pump 218 produces, monitoring the motor current and controlling this current to the motor, provide for monitoring and controlling, respectively, the negative pressure produced by the vacuum pump.

Motor current monitoring is only one method for indirect negative pressure monitoring and controlling. Alternatively or additionally, the motor parameter being monitored may be the torque and/or speed of the motor, which in turn are also related to the negative pressure generated by the pump. Thus, a constant torque level or rpm may be provided by adjusting the level of the torque clutch accordingly, and/or, a torque sensor may be provided, operatively connected to an alarm, to alert the user when the torque level or rpm (and thus the negative pressure at the wound enclosure 212) drops below a predetermined value.

The same function of negative pressure control may be accomplished by an adjustable torque limiting clutch placed between the motor output shaft and the crank 227. When the desired pre-set vacuum level is reached, the clutch will start slipping and prevent any excess motor torque from generating excess negative pressure at the target volume.

The control block 350 comprises a negative pressure comparator 344, which compares the desired set negative pressure level obtained by pump 218, and the actual monitored negative pressure level as obtained indirectly from motor voltage and current monitoring and control unit 343. Comparator 344 will activate audible alarm 345, whenever pump 218 fails to reach the desired pre-set negative pressure level. Optionally, the control block 350 may comprise a display for displaying, for example digitally, or graphically as a function of time, the vacuum level at the target volume, as derived from the drive unit parameter that is being monitored.

The components of the control block 350 may comprise discrete electronic components operatively interconnected to operate as described herein; alternatively, control block 350 may comprise a suitable microprocessor unit, programmed with suitable software, and operatively coupled to the to the drive unit.

While the monitor/control system 300 has been described above in the context of the fourth embodiment, the system 300 may be comprised in the other embodiments disclosed herewith in a similar manner thereto, mutatis mutandis. Furthermore, the system 300 may also be used in conjunction with other vacuum pumping systems in which it is desired to monitor and control the suction pressure thereof, while minimizing risk of contamination to or from the fluid being pumped therethrough.

Some embodiments of the invention are defined in the following numbered paragraphs:
1. A vacuum system for providing a sub-ambient pressure to a target volume, comprising:
   a suction head having an inlet arrangement adapted for being in fluid communication with said target volume;
   a vacuum pump in fluid communication with said suction head, said vacuum pump being adapted for providing a working pressure in said system below external ambient pressure;
   a waste container defining a collection volume for collection of materials that may be drained from said target volume in fluid communication with at least one of said vacuum pump and said inlet arrangement; and
   a passive pressure regulation system for passively regulating said working pressure, said passive pressure regulation system being in fluid communication with said suction head upstream of the said waste container.
2. A vacuum system according to embodiment 1, wherein said pressure regulation system comprises a venting valve arrangement adapted for enabling ingress of external ambient air into the system responsive to a reduction in said working pressure below a predetermined datum pressure with respect to said external ambient pressure and for discontinuing said ingress when said datum pressure is restored.
3. A vacuum system according to embodiment 2, wherein said venting valve arrangement comprises an inlet port having a valve seat and in fluid communication with said ambient air, an outlet port in fluid communication with said vacuum system, and a valve seal biased for sealingly closing with respect to said valve seat by means of a resilient element generating a biasing force of magnitude substantially less than and in a direction generally opposed to a pressure-induced force acting on said valve seal when said working pressure is less than said datum pressure.
4. A vacuum system according to embodiment 2, wherein said venting valve arrangement comprises an adjustment mechanism for adjusting said datum pressure.
5. A vacuum system according to embodiment 4, wherein said venting valve arrangement comprises an inlet port in fluid communication with said ambient air and an outlet port in fluid communication with said vacuum system, and a valve seal biased for sealingly closing said inlet port by means of a resilient element generating a biasing force of magnitude substantially less than and in a direction generally opposed to a pressure-induced force acting on said valve seal when said working pressure is less than said datum pressure, and wherein said adjustment mechanism comprises a bias adjusting arrangement for adjusting the magnitude of said biasing force.
6. A vacuum system according to embodiment 5, wherein said resilient element comprises a compression spring mounted in a suitable housing such as to urge said valve seal towards said valve seat, and said bias adjusting arrangement comprises a compression control mechanism for adjusting the compression of said spring.
7. A vacuum system according to embodiment 1, wherein said pressure regulation system is connected to said suction head permitting selective fluid communication between said target volume and said external ambient air, enabling ingress of external ambient air into said target volume responsive to a reduction in said working pressure below a predetermined datum pressure and discontinuing said ingress when said datum pressure is restored.
8. A vacuum system according to embodiment 7, wherein said pressure regulation system is connected to said suction head via a suitable conduit.
9. A vacuum system according to embodiment 8, wherein said pressure regulation system is in selective fluid communication with an outlet port of said vacuum pump and said external ambient air such as to allow ingress of at least one of fluid from said vacuum pump outlet port and said external ambient air when said working pressure is below said datum pressure.
10. A vacuum system according to embodiment 1, wherein said vacuum pump comprises a pump head releasably operatively coupled to a pump drive unit, wherein said pump head is one of fixed to and integral with said waste container.
11. A vacuum system according to embodiment 10, wherein said pump head comprises a pump inlet port and a pump outlet port, and further comprises a suitable deformable diaphragm arrangement, reciprocable by means of said pump drive unit, to induce said working pressure in said vacuum system.
12. A vacuum system according to embodiment 11, wherein said drive unit comprises a reciprocation drive for driving a reciprocating member, and said diaphragm is releasably engageble with said reciprocating member responsive said pump head being coupled to said pump unit.
13. A vacuum system according to embodiment 12, wherein said reciprocating member and said diaphragm are conformally shaped such that when coupled one with the other a vacuum is created therebetween.
14. A vacuum system according to embodiment 11, wherein a part of said pump head is accommodated in said waste container, wherein said diaphragm is facing a direction generally away from said collection volume, and wherein said pump inlet port and a pump outlet port are at least partially within said collection volume.
15. A vacuum system according to embodiment 14, wherein said pump inlet port is in fluid communication with said suction head via a conduit, said pump outlet port is in fluid communication with said collection volume and wherein said waste container is vented to said external ambient air.
16. A vacuum system according to embodiment 14, wherein said pump inlet port is in fluid communication with said suction head via said collection volume, and said pump outlet port is vented to said external ambient air.
17. A vacuum system according to embodiment 16, wherein said pressure regulation system is operatively connected to said suction head via a suitable first conduit and wherein said pressure regulation system is in selective fluid communication with said pump outlet port and said external ambient air such as to allow ingress of at least one of fluid from said vacuum pump outlet port and external ambient air into said target volume responsive to a reduction in said working pressure below a predetermined datum pressure and such as to discontinue said ingress when said datum pressure is restored.
18. A vacuum system according to embodiment 17, wherein said waste container is in fluid communication with said suction head via a suitable second conduit.
19. A vacuum system according to embodiment 18, wherein said first conduit and said second conduit are in fluid communication one with another.
20. A vacuum system according to embodiment 14 or embodiment 73, wherein said pump head, waste container and suction head are configured for being disposable.
21. A vacuum system according to embodiment 1, wherein said target volume is associated with a wound, burn or the like, and said suction head comprises an enclosure sealable to a perimeter of the wound so as to define a confined volume comprising said target volume.
22. A vacuum system according to embodiment 1, wherein said suction head comprises a drain catheter having at least one lumen in fluid communication with said pump, and said inlet arrangement comprises at least one aperture adapted for providing fluid communication between said target volume and said at least one lumen.
23. A vacuum system for providing a sub-ambient pressure to a medical target volume, comprising:
a suction head having an inlet arrangement adapted for being in fluid communication with said target volume;
a vacuum pump in fluid communication with said suction head, said vacuum pump being adapted for providing a working pressure in said system below external ambient pressure, said vacuum pump comprising a pump head releasably operatively connected to a pump drive unit, wherein said pump head comprises a pump inlet port, a pump outlet port and a suitable deformable diaphragm arrangement reciprocable by means of said pump drive unit to induce said working pressure in said vacuum system;
a waste container defining a collection volume for enabling collection of drained materials from said target volume, wherein said pump head is one of fixed to and integral with said waste container such that said pump inlet port and said pump outlet port are accommodated in said collection volume, wherein said pump inlet port is in fluid communication with said suction head via a conduit connecting said pump inlet port with said suction head, at least a portion of said conduit being accommodated in said collection volume, wherein said pump outlet port discharges into said collection volume, and wherein said waste container is vented to external ambient air; and
a venting valve arrangement mounted to one of said conduit and suction head adapted for enabling ingress of external ambient air into the system responsive to a reduction in said working pressure below a predetermined datum pressure with respect to said external ambient pressure, such as to induce suction flow of materials from said target volume when in use, and for discontinuing said ingress when said datum is pressure is restored.
24. A vacuum system according to embodiment 23, wherein said pump head and waste container are reversibly lockably engaged with said pump drive unit by means of a latch arrangement
25. A vacuum system for providing a sub-ambient pressure to a medical target volume, comprising:
a suction head having an inlet arrangement adapted for being in fluid communication with said target volume;
a vacuum pump in fluid communication with said suction head, said vacuum pump being adapted for providing a working pressure in said system below external ambient pressure, said vacuum pump comprising a pump head releasably operatively connected to a pump drive unit, wherein said pump head comprises a pump inlet port, a pump outlet port and a suitable deformable diaphragm arrangement reciprocable by means of said pump drive unit to induce said working pressure in said vacuum system;
a waste container defining a collection volume for enabling collection of drained materials from said target volume, wherein said pump head is one of fixed to and integral with said waste container such that said pump inlet port and said pump outlet port are accommodated in said collection volume, wherein said pump inlet port is in fluid communication with said suction head via said collection volume, and said waste container is in fluid communication with said suction head via a first conduit, and wherein said pump outlet port is vented to external ambient air; and
a venting valve arrangement in fluid communication with said suction head via a second conduit, said valve arrangement being mounted to said waste container and in selective fluid communication with said pump outlet port for enabling ingress of at least one of fluid from said vacuum pump outlet port and external ambient air into the system responsive to a reduction in said working pressure below a predetermined datum pressure with respect to said external ambient pressure, such as to induce suction flow of materials from said target volume when in use, and for discontinuing said ingress when said datum pressure is restored.
26. A vacuum system according to embodiment 25, wherein said pump head and waste container are reversibly lockably engaged with said pump drive unit by means of a latch arrangement
27. A method for providing a sub-ambient pressure to a medical target volume, comprising:
inducing a working pressure in said target volume below external ambient pressure;
providing a collection volume for collection of drained materials from said target volume; and
regulating the working pressure in said target volume to provide a positive pressure gradient between said target volume and said collection volume such as to aid flow of said materials from said target volume to said collection volume.
28. Method according to embodiment 27, wherein step (c) comprises allowing ingress of external ambient air at or near to the target volume responsive to a reduction in said working pressure below a predetermined datum pressure with respect to said external ambient pressure and discontinuing said ingress when said datum pressure is restored.
29. Method according to embodiment 28, further comprising allowing recirculation of fluid between said collection volume and said target volume together with said ingress of external ambient air.
30. A kit for use with a vacuum system for providing a sub-ambient pressure to a medical target volume, comprising:
a vacuum pump head adapted for releasable operative connection to a pump drive unit, said pump head comprising a pump inlet and a pump outlet for enabling working fluid to be pumped through the pump during operation thereof;
a waste container defining a collection volume for collection of drained materials in fluid communication with at least one of said vacuum pump head;
wherein said vacuum pump head is one of attached to or integral with said waste container such that at least one of said pump inlet and said pump outlet is accommodated in said collection volume.
31. A kit according to embodiment 30, further comprising a suction head having an inlet arrangement adapted for being in fluid communication with said target volume.
32. A kit according to embodiment 30, wherein said suction head comprises an enclosure sealable to a perimeter of a wound so as to define a confined volume comprising said target volume, said enclosure being in fluid communication with at least one of said waste container and said pump inlet.
33. A kit according to embodiment 30, wherein said suction head comprises a drain catheter having at least one lumen in fluid communication with said pump, and said inlet arrangement comprises at least one aperture adapted for providing fluid communication between said target volume and said at least one lumen, said drain catheter being in fluid communication with at least one of said waste container and said pump inlet.
34. A kit according to embodiment 30, further comprising a passive pressure regulation system for regulating said working pressure.
35. A kit according to embodiment 30, comprising:
a suction head having an inlet arrangement adapted for being in fluid communication with said target volume;
a pump head adapted for being releasably operatively connected to a pump drive unit, said pump head comprising a pump inlet port, a pump outlet port and a suitable deformable diaphragm arrangement adapted for reciprocable operation by means of said pump drive unit when connected thereto;
a waste container defining a collection volume for collection of drained materials from said target volume, wherein said pump head is one of fixed to or integral with said waste container such that said pump inlet port and said pump outlet port are accommodated in said collection volume, wherein said pump inlet port is in fluid communication with said suction head via a conduit connecting said pump inlet port with said suction head, at least a portion of said conduit being accommodated in said collection volume, wherein said pump outlet port is adapted for discharging into said collection volume, and wherein said waste container is vented to external ambient air; and
a venting valve arrangement mounted to one of said conduit and suction head adapted for enabling ingress of external ambient air into the system responsive to a reduction in said working pressure below a predetermined datum pressure with respect to said external ambient pressure, such as to induce suction flow of materials from said target volume when in use, and for discontinuing said ingress when said datum pressure is restored.
36. A vacuum system according to embodiment 35, wherein said diaphragm comprises a suction cup arrangement for engagement with said pump drive unit.
37. A kit according to embodiment 30, comprising:
a suction head having an inlet arrangement adapted for being in fluid communication with said target volume;
a pump head adapted for being releasably operatively connected to a pump drive unit, said pump head comprising a pump inlet port, a pump outlet port and a suitable deformable diaphragm arrangement adapted for reciprocable operation by means of said pump drive unit when connected thereto;
a waste container defining a collection volume for collection of drained materials, wherein said pump head is fixed to said waste container such that said pump inlet port and said pump outlet port are accommodated in said collection volume, wherein said pump inlet port is in fluid communication with said suction head via said collection volume, and said waste container is in fluid communication with said suction head via a first conduit, and wherein said pump outlet port is vented to external ambient air; and
a venting valve arrangement in fluid communication with said target volume via a second conduit, said valve arrangement being mounted to said waste container and in selective fluid communication with said pump outlet port for enabling ingress of at least one of fluid from said vacuum pump outlet port and external ambient air into the system responsive to a reduction in said working pressure below a predetermined datum pressure with respect to said external ambient pressure, such as to induce suction flow of materials from said target volume, and for discontinuing said ingress when said datum pressure is restored.
38. A vacuum system according to embodiment 37, wherein said diaphragm comprises a suction cup arrangement for engagement with said pump drive unit.
40. A vacuum system for draining an open wound from liquids exuded therefrom, comprising an enclosure sealable to the wound circumference so as to define a confined volume, a vacuum pump in fluid communication with said confined volume, and a waste container for collection of drained liquids in fluid communication with said vacuum pump,
wherein said confined volume is connected to an inlet of said vacuum pump and said waste container is connected to an outlet of said vacuum pump, such that when said vacuum pump is operated the drained liquids flow through said vacuum pump.
41. The vacuum system of embodiment 40, wherein said enclosure has an outlet connected by means of a tube to said inlet of the vacuum pump, and one or more bleeding holes are provided through said enclosure or adjacent to its outlet so that ambient air may enter said tube and flow together with the drained liquids.
42. The vacuum system of embodiments 40 or 41, wherein said vacuum pump includes a disposable pump unit detachably attachable to a non-disposable drive unit, said enclosure and said waste container being disposable so that the drained liquids, as well as air coming in contact with them, contact only the disposable elements and said drained liquids may be disposed of together with said disposable elements.
43. The vacuum system of embodiment 42, wherein said pump unit and said drive unit are adapted for attaching and detaching by simple hand manipulations.
44. The vacuum system of any one of embodiments 40 to 43, wherein said vacuum pump includes a drive unit and a control block adapted to power said drive unit so that a predetermined level of negative pressure is maintained in said confined volume,
wherein said control block has a sensor for sensing working parameters of said drive unit and means for deriving the level of negative pressure in said confined volume from said working parameters, in order to maintain said predetermined level, said sensor having no fluid communication with said confined volume.
45. The vacuum system of embodiment 44, wherein said drive unit comprises an electric motor and said working parameters include the electric current and/or voltage of said motor.
46. The vacuum system of any one of embodiments 40 to 45, wherein said waste container is vented so as to release gases mixed with said drained liquids.
47. The vacuum system of any one of embodiments 40 to 46, wherein said waste container is a collapsible bag.
48. The vacuum system of any one of embodiments 40 to 47, wherein said vacuum pump is a two-chambered diaphragm pump adapted for pumping gases and liquids and/or any combination thereof.
49. The vacuum system of any one of embodiments 40 to 48, adapted to be worn by an ambulatory patient.
50. An enclosure for draining an open wound from liquids exuded therefrom, the enclosure being attachable to the wound circumference so as to define a confined volume, and having an outlet connectable by means of a tube to an inlet of a vacuum pump so that negative pressure may be created in said volume,
wherein one or more bleeding holes are provided in the enclosure or adjacent to its outlet such that ambient air can enter said tube and flow together with the exuded liquids under the action of the negative pressure, and the ambient pressure may be restored in said confined volume when said vacuum pump is not operating.
51. The enclosure of Embodiment 50, having a nipple for connecting to said tube.
52. The enclosure of any one of embodiments 50 to 51, wherein a plurality of bleeding holes is provided in the form of a porous body mounted in said enclosure.
53. The enclosure of any one of embodiments 50 to 52, completed with a tube connected to said outlet, for connection to said vacuum pump inlet, wherein said one or more bleeding holes are provided in the tube, adjacent to said outlet.
54. A method for draining an open wound from liquids exuded therefrom, including: providing an enclosure and sealing it to the wound circumference so as to define a confined volume,
connecting said confined volume to a vacuum pump,
connecting a waste container for collection of drained liquids to said vacuum pump,
operating said vacuum pump to drain the exuded liquids,
wherein
said confined volume is connected to an inlet of said vacuum pump and said waste container is connected to an outlet of said vacuum pump such that the drained liquids and air flow through said vacuum pump.
55. The method of embodiment 54, wherein said enclosure has an outlet connected by means of a tube to said inlet of the vacuum pump, and ambient air is allowed to enter into said tube via one or more bleeding holes provided through said enclosure or adjacent to its outlet so that said air flows together with the drained exuded liquids.
56. The method of any one of embodiments 54 to 55, comprising separating and releasing of gases from the drained exuded liquids.
57. The method of embodiment 56, wherein said gases are released through a vent in the waste container.
58. The method of any one of embodiments 54 to 57, wherein said vacuum pump is a two-chambered diaphragm pump adapted for pumping gases and liquids and/or any combination thereof
59. The method any one of embodiments 54 to 58, wherein said enclosure, said waste container, and at least a part of said vacuum pump which contacts said drained liquids, are disposable, and the method comprises disposing of the disposable elements together with the drained exuded liquids.
60. The method of embodiment 59, wherein the disposable part of said vacuum pump is a pump unit detachably attachable to a non-disposable drive unit, the method including attaching the pump unit to the drive unit before draining, and detaching the pump unit before said disposing.
61. The method of embodiment 60, wherein said attaching and detaching include only simple hand manipulations.
62. The method any one of embodiments 54 to 61, applied to a wound of an ambulatory patient.
63. A disposable assembly for draining an open wound from liquids exuded therefrom, the assembly comprising an enclosure attachable to the wound circumference so as to define a confined volume, a vacuum pump unit connected to said enclosure so that negative pressure may be created in said volume, said vacuum pump unit having means for detachably attaching to a drive unit for operating the pump unit, and a waste container connected to said vacuum pump unit;
wherein said enclosure is connected to an inlet of said vacuum pump unit and said waste container is connected to an outlet of said vacuum pump unit, such that when said vacuum pump unit is operated the drained liquids flow therethrough.
64. The disposable assembly of embodiment 63, wherein said enclosure has an outlet connected by means of a tube to said inlet of the vacuum pump unit, and one or more bleeding holes are provided through said enclosure or adjacent to its outlet so that ambient air may flow through said tube together with the drained exuded liquids.
65. The disposable assembly of any one of embodiments 63 to 64, wherein said vacuum pump unit is a two-chambered diaphragm pump adapted for pumping gases and liquids and/or any combination thereof.
66. The disposable assembly of any one of embodiments 63 to 65, wherein said waste container contains a porous media adapted to soak up said drained liquids.
67. The disposable assembly of any one of embodiments 63 to 66, wherein said waste container is a collapsible bag.
68. A vacuum system for draining an open wound from liquids exuded therefrom, comprising an enclosure sealable to the wound circumference so as to define a confined volume, a vacuum pump in fluid communication with said confined volume, said vacuum pump having a drive unit, and a control block adapted to power said drive unit so that a predetermined level of negative pressure is maintained in said confined volume,
wherein
said control block has a sensor for sensing working parameters of said drive unit and means for deriving the level of negative pressure in said confined volume from said working parameters, in order to maintain said predetermined level, said sensor having no fluid communication with said confined volume.
69. The vacuum system of embodiment 68, wherein said drive unit comprises an electric motor and said working parameters include the electric current and/or voltage of said motor.
70. The vacuum system of any one of embodiments 68 to 69, wherein said control block has alarm means to warn the user if said predetermined level of negative pressure is not maintained.
71. The vacuum system of any one of embodiments 68 to 70, further comprising a waste container for collection of drained liquids, where said confined volume is in fluid communication with an inlet of said vacuum pump, and said waste container is in fluid communication with an outlet of said vacuum pump such that when said vacuum pump is operated the drained liquids flow therethrough.
72. The vacuum system of any one of embodiments 68 to 71, wherein said vacuum pump comprises a disposable pump unit including said inlet and said outlet, and said pump unit is detachably attachable to said drive unit, said control block with said sensor being associated with the drive unit,
73. A vacuum system for providing a sub-ambient pressure to a target volume, comprising:
a suction head having an inlet arrangement adapted for being in fluid communication with said target volume;
a vacuum pump comprising a pump head releasably connected to a powered pump drive, said pump head being in fluid communication with said suction head, said vacuum pump being adapted for providing a working pressure in said system below external ambient pressure;
a waste container defining a collection volume for collection of materials that may be drained from said target volume, said waste container being in fluid communication with at least one of said vacuum pump and said inlet arrangement, wherein said waste container is integrated with said pump head.
a passive pressure regulation system for passively regulating said working pressure, said passive pressure regulation system being in fluid communication with said suction head upstream of the said waste container.

and wherein said enclosure and said waste container are disposable, so that said exuded liquids may contact only the disposable elements of the vacuum system and may be disposed of together with said disposable elements after detaching said drive unit.

In the method claims that follow, alphanumeric characters and Roman numerals used to designate claim steps are provided for convenience only and do not imply any particular order of performing the steps.

It should be noted that the word “comprising” as used throughout the appended claims is to be interpreted to mean “including but not limited to”.

While there has been shown and disclosed example embodiments in accordance with the invention, it will be appreciated that many changes may be made therein without departing from the spirit of the invention.

1. A vacuum system for providing a sub-ambient pressure to a target volume, comprising:
a suction head having an inlet arrangement adapted for being in fluid communication with said target volume;
a vacuum pump in fluid communication with said suction head, said vacuum pump being adapted for providing a predetermined working pressure in said system below external ambient pressure of an external environment with respect to said suction head;
a waste container defining a collection volume for collection of materials that may be drained from said target volume in fluid communication with at least one of said vacuum pump and said inlet arrangement; and
a passive pressure regulation system for maintaining said working pressure, said passive pressure regulation system being in fluid communication with said suction head upstream of the said waste container, and said passive pressure regulation system being in one of selective and permanent fluid communication with said external environment.

2. A vacuum system according to claim 1, wherein said pressure regulation system comprises a venting valve arrangement adapted for enabling ingress of external ambient air into the system responsive to a reduction in said working pressure below a predetermined datum pressure with respect to said external ambient pressure and for discontinuing said ingress when said datum pressure is restored.

3. A vacuum system according to claim 2, wherein said venting valve arrangement comprises an inlet port having a valve seat and in fluid communication with said ambient air, an outlet port in fluid communication with said vacuum system, and a valve seal biased for sealingly closing with respect to said valve seat by means of a resilient element generating a biasing force of magnitude substantially less than and in a direction generally opposed to a pressure-induced force acting on said valve seal when said working pressure is less than said datum pressure.

4. A vacuum system according to claim 2, wherein said venting valve arrangement comprises an adjustment mechanism for adjusting said datum pressure.

5. A vacuum system according to claim 1, wherein said pressure regulation system is connected to said suction head permitting selective fluid communication between said target volume and said external ambient air, enabling ingress of external ambient air into said target volume responsive to a reduction in said working pressure below a predetermined datum pressure and discontinuing said ingress when said datum pressure is restored.

6. A vacuum system according to claim 5, wherein said pressure regulation system is in selective fluid communication with an outlet port of said vacuum pump and said external ambient air such as to allow ingress of at least one of fluid from said vacuum pump outlet port and said external ambient air when said working pressure is below said datum pressure.

7. A vacuum system according to claim 1, wherein said pressure regulation system comprises a venting arrangement adapted for providing, at least during operation of said system, substantially permanent fluid communication between said suction head and at least one of said external environment, such as to enable said working pressure to be maintained at the suction head while enabling a desired flow rate of ambient air into the suction head via said venting arrangement, and an irrigation source, to enable irrigation of said target volume with a desired irrigation material.

8. A vacuum system according to claim 7, wherein said venting arrangement comprises at least one bleeding orifice comprising an effective flow area compatible with providing said desired flow rate.

9. A vacuum system according to claim 1, wherein said vacuum pump comprises a pump head releasably operatively coupled to a pump drive unit, wherein said pump head is one of fixed to and integral with said waste container.

10. A vacuum system according to claim 2, wherein said vacuum pump comprises a pump head releasably operatively coupled to a powered pump drive unit, wherein said pump head is one of fixed to and integral with said waste container.

11. A vacuum system according to claim 10, wherein said pump head comprises a pump inlet port and a pump outlet port, and further comprises a suitable deformable diaphragm arrangement, reciprocable by means of said pump drive unit, to induce said working pressure in said vacuum system.

12. A vacuum system according to claim 11, wherein said drive unit comprises a reciprocation drive for driving a reciprocating member, and said diaphragm is releasably engageble with said reciprocating member responsive said pump head being coupled to said pump unit.

13. A vacuum system according to claim 12, wherein said reciprocating member and said diaphragm are conformally shaped such that when coupled one with the other a vacuum is created therebetween.

14. A vacuum system according to claim 11, wherein a part of said pump head is accommodated in said waste container, wherein said diaphragm is facing a direction generally away from said collection volume, and wherein said pump inlet port and a pump outlet port are at least partially within said collection volume.

15. A vacuum system according to claim 14, wherein said pump inlet port is in fluid communication with said suction head via a conduit, said pump outlet port is in fluid communication with said collection volume and wherein said waste container is vented to said external ambient air.

16. A vacuum system according to claim 14, wherein said pump inlet port is in fluid communication with said suction head via said collection volume, and said pump outlet port is vented to said external ambient air.

17. A vacuum system according to claim 16, wherein said pressure regulation system is operatively connected to said suction head via a suitable first conduit and wherein said pressure regulation system is in selective fluid communication with said pump outlet port and said external ambient air such as to allow ingress of at least one of fluid from said vacuum pump outlet port and external ambient air into said target volume responsive to a reduction in said working pressure below a predetermined datum pressure and such as to discontinue said ingress when said datum pressure is restored.

18. A vacuum system according to claim 17, wherein said waste container is in fluid communication with said suction head via a suitable second conduit.

19. A vacuum system according to claim 18, wherein said first conduit and said second conduit are in fluid communication one with another.

20. A vacuum system according to claim 14, wherein said pump head, waste container and suction head are configured for being disposable.

21. A vacuum system according to claim 1, wherein said target volume is a medical target volume associated with a wound, burn or the like, and said suction head comprises an enclosure sealable to a perimeter of the wound burn or the like, respectively, so as to define a confined volume comprising said target volume.

22. A vacuum system according to claim 1, further comprising a non fluid invasive monitoring system for monitoring said working pressure, said monitoring system comprising at least one sensor for monitoring a working parameter of said powered pump and a comparator unit for comparing monitored data corresponding to said working parameter with threshold data, wherein the or each said working parameter is directly related to a magnitude of said working pressure provided by said vacuum pump.

23. A vacuum system for providing a sub-ambient pressure to a target volume, comprising:
a suction head having an inlet arrangement adapted for being in fluid communication with said target volume;
a vacuum pump in fluid communication with said suction head, said vacuum pump being adapted for providing a predetermined working pressure in said system below external ambient pressure of an external environment with respect to said suction head;
a first waste container defining a collection volume for collection of materials that may be drained from said target volume, said first waste container being coupled to an upstream part of said vacuum pump; and
a second waste container defining a collection volume for collection of materials that may be drained from said target volume, said second waste container being coupled to a downstream part of said vacuum pump.

24. A vacuum system comprising a vacuum pump adapted for providing a predetermined working pressure in said system below a reference pressure, said vacuum pump comprising a pump head operatively coupled to a powered pump drive unit, and further comprising a non fluid invasive monitoring system for monitoring said working pressure, said monitoring system comprising at least one sensor for monitoring a working parameter of said powered pump and a comparator unit for comparing monitored data corresponding to said working parameter with threshold data, wherein the or each said working parameter is directly related to a magnitude of said working pressure provided by said vacuum pump.

25. A vacuum system according to claim 24, wherein said monitoring system comprises a suitable alarm configured for being activated when a magnitude of said monitored data exceeds or is below a magnitude of said threshold data.

26. A vacuum system according to claim 24, wherein said monitoring system comprises a display for displaying said monitored data in any suitable manner.

27. A vacuum system according to claim 24, wherein said drive unit comprises an electric motor, and at least one said parameter comprises any one of: motor current, motor voltage, motor power, motor rotational speed, motor torque.

28. A vacuum system according to claim 1, further comprising:
a first waste container defining a collection volume for collection of materials that may be drained from said target volume, said first waste container being coupled to an upstream part of said vacuum pump; and
a second waste container defining a collection volume for collection of materials that may be drained from said target volume, said second waste container being coupled to a downstream part of said vacuum pump.

29. A kit for use with a vacuum system for providing a sub-ambient pressure to a target volume, comprising:
a vacuum pump head adapted for releasable operative connection to a powered pump drive unit, said pump head comprising a pump inlet and a pump outlet for enabling working fluid to be pumped through the pump during operation thereof;
a waste container defining a collection volume for collection of drained materials in fluid communication with at least one of said vacuum pump head;
wherein said vacuum pump head is one of attached to or integral with said waste container such that at least one of said pump inlet and said pump outlet is accommodated in said collection volume.

30. A kit according to claim 29, further comprising a suction head having an inlet arrangement adapted for being in fluid communication with said target volume.

31. A kit according to claim 29, wherein said suction head comprises an enclosure sealable to a perimeter of a wound so as to define a confined volume comprising said target volume, said enclosure being in fluid communication with at least one of said waste container and said pump inlet.

32. A kit according to claim 29, further comprising a passive pressure regulation system for regulating said working pressure.

33. A method for providing a sub-ambient pressure to a target volume, comprising:
(a) inducing a working pressure in said target volume below external ambient pressure;
(b) providing a collection volume for collection of drained materials from said target volume; and
(c) regulating the working pressure in said target volume to maintain a positive pressure gradient between said target volume and said collection volume such as to aid flow of said materials from said target volume to said collection volume.

34. Method according to claim 33, wherein step (c) comprises allowing ingress of external ambient air to the target volume responsive to a reduction in said working pressure below a predetermined datum pressure with respect to said external ambient pressure and discontinuing said ingress when said datum pressure is restored.

35. Method according to claim 34, further comprising allowing recirculation of fluid between said collection volume and said target volume together with said ingress of external ambient air.

36. Method according to claim 33, wherein step (c) comprises selectively allowing substantially continuous ingress of external ambient air to the target volume, such as to enable said working pressure to be maintained at the target volume while enabling a desired flow rate of ambient air into the target volume.

37. Method according to claim 36, comprising the step of providing a pulsating pressure to said target volume by alternately inducing said working pressure at said target volume and alternately venting said target volume to external ambient pressure.

38. A method for indirectly monitoring a working pressure generated by a vacuum pump in a vacuum system, said working pressure being below a reference pressure, said vacuum pump comprising a pump head operatively coupled to a powered pump drive unit, the method comprising monitoring a working parameter of said powered pump and comparing monitored data corresponding to said working parameter with threshold data, wherein the or each said working parameter is directly related to a magnitude of said working pressure provided by said vacuum pump.

39. A method according to claim 38, further comprising activating an alarm when a magnitude of said monitored data exceeds or is below a magnitude of said threshold data.

40. A vacuum system according to claim 38, further comprising displaying said monitored data in any suitable manner.

41. A vacuum system according to claim 38, wherein at least one said parameter comprises any one of: motor current, motor voltage, motor power, motor torque.

42. A vacuum system for providing a sub-ambient pressure to a target volume, comprising:
a suction head having an inlet arrangement adapted for being in fluid communication with said target volume;
a vacuum pump in fluid communication with said suction head, said vacuum pump being adapted for providing a predetermined working pressure in said system below external ambient pressure of an external environment with respect to said suction head;
a waste container defining a collection volume for collection of materials that may be drained from said target volume in fluid communication with an outlet port of said vacuum pump; and
a passive pressure regulator for maintaining said working pressure, said passive pressure regulation system having an outlet in fluid communication with said suction head upstream of the said waste container, and said passive pressure regulator system having an inlet in selective fluid communication with said collection volume.

43. A vacuum system according to claim 42, wherein said pressure regulator comprises a venting valve arrangement adapted for enabling ingress of external ambient air into the system responsive to a reduction in said working pressure below a predetermined datum pressure with respect to said external ambient pressure and for discontinuing said ingress when said datum pressure is restored.

44. A vacuum system according to claim 43, wherein said venting valve arrangement comprises an inlet port having a valve seat in selective fluid communication with said ambient air, and a valve seal biased for sealingly closing with respect to said valve seat by means of a resilient element generating a biasing force of magnitude substantially less than and in a direction generally opposed to a pressure-induced force acting on said valve seal when said working pressure is less than said datum pressure.

45. A vacuum system according to claim 42, wherein said pump comprises a pump head reversibly engageable with a powered pump drive unit, and wherein said container is integral with said pump head.
